(19) United States
(12) Patent Application Publication
(10) Pub. No.: US 2012/0029471 A1
(43) Pub. Date: Feb. 2, 2012

(54) TWO CHAMBER SYRINGE WITH LOCKING MECHANISM

(76) Inventors: Martin N. Lee, Sacramento, CA (US); Goeffrey H. Willis, Santa Cruz, CA (US); Jeffrey J. Christian, Morgan Hill, CA (US)

(21) Appl. No.: 12/847,825

(22) Filed: Jul. 30, 2010

(51) Int. Cl. A61M 5/31

(52) U.S. Cl. 604/518; 604/85

(57) ABSTRACT

A syringe includes a cartridge and a second chamber. The cartridge includes a first chamber, a second end, and a locking mechanism. The second end is movable within the first chamber between a first position and a second position. The locking mechanism has a locked configuration and an unlocked configuration and prevents movement of the second end within the first chamber while in the locked configuration. The locking mechanism includes: a flexible arm having a first end coupled to the second end of the cartridge and a second free end; a tab coupled to the second free end; and a groove in the inner surface of the chamber configured to receive the tab. The cartridge is moveable within the second chamber.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

1. Field of the Invention

This invention relates to syringe injectors.

2. General Background

Syringes are commonly used in the medical field for the injection or withdrawal of liquid medications. Syringes typically have a hollow glass or plastic barrel with an internal piston. By moving the piston, a user can create a positive or negative pressure inside the barrel, thereby transmitting fluid out of or into the barrel through a small opening opposite the piston.

Syringes are often used in intravenous therapy where the syringe may directly puncture the vein, or more commonly, may be used in conjunction with a catheter. When a catheter is used, one side of the catheter remains in the vein, while the other side remains outside the skin. The external portion of the catheter typically includes a coupler for connection to a syringe.

After injection in either procedure, a small amount of medication is typically left behind. When a syringe is used, the medication remains within the tip of the syringe. When a catheter is used in conjunction with a syringe, the unadministered medication remains in both the tip of the syringe and in the catheter.

This leftover medication is problematic for several reasons. First, it necessarily means that the entire amount of medicine drawn into the syringe does not reach the patient. Second, many medications are time sensitive and should not remain in the catheter until a subsequent medicine flushes it through.

In a catheter system, these problems are solved using a second liquid to immediately flush the remaining medication out of the catheter and into the patient. Generally, a second syringe prefilled with a flushing solution provides the second liquid.

While many different liquids may be used to flush the catheter, the most commonly used liquid is a 0.9% concentration of sodium chloride (saline solution). The saline solution is injected from a syringe into the catheter, thereby flushing any stranded medication into the patient. Thus, the saline flush ensures that a full dosage of medication has been timely delivered.

This method for purging the catheter has certain disadvantages. For instance, by using a separate syringe for each injection, there is an increased chance of medical error. Most medicines are colorless (like the saline solution), and it is easy to accidentally administer medication when intending to flush the line or vice versa. This risk is increased when clinicians carry medicines for multiple patients at one time.

The likelihood of error is compounded in an emergency, when it may be necessary to inject several medications quickly and in a specific order. In such situations, a separate saline flush is necessary between every individual medication injection, so the risk of error is high, and the consequences of a mistake may be grave.

Finally, the clinician may be distracted by a separate medical need during the time between the injection of medication and the saline flush. Without some reminder, the clinician may forget that he or she has not flushed the line.

Even if all precautions are taken and the two injections are made in the proper order, drawbacks remain. With each breach of the catheter's seal for injection, the patient is potentially exposed to bacteria, increasing the risk of infection. By requiring a clinician to access the system once for the medication and a second time for the flush, the risk of infection is doubled.

Using a second syringe for the saline flush also wastes resources. Attaching a second syringe to the catheter takes time, and since a clinician may perform a saline flush more than one hundred times per day, this lost time adds up quickly. Finally, requiring a second syringe unnecessarily increases the already significant costs related to manufacturing, shipping, storage, and disposal of syringes.

Syringes adapted to deliver multiple fluids for sequential injection have been described in the prior art. However, due to design limitations, no syringe has become widely accepted that allows routine medication administration and subsequent catheter flushing from a single syringe. Some prior art syringes include a “standard” syringe that is separated by an intermediate sliding stopper into two chambers. The sliding stopper receives motive force communicated through an intermediate fluid from a primary stopper (part of a plunger assembly of the standard syringe) against which an external force is applied. Examples of such prior art devices may be found in U.S. Pat. Nos. 5,720,731, 6,997,910 and 7,101,354, which describe multiple embodiments of a conventional syringe adapted to deliver multiple fluids and a displaceable valved stopper which partitions a conventional syringe. Other sequential delivery syringes have been developed, such as U.S. Pat. No. 6,723,074, which uses a piercing member to open an internal chamber.

The previously described syringes adapted to deliver multiple fluids have not been widely adopted because they generally require the delivery of two fluids that are both prefilled during manufacturing. Rather, current standard practice is to use two separate syringes—one prefilled with saline and one empty syringe that is filled with medication shortly before administration. Requiring hospitals to change their practice to using previously described multi-fluid syringes is unfeasible because the hospitals would be required to keep an unreasonable number of prefilled medicine syringes in stock to accommodate the varied number of doses and types of medications required for routine patient care. The applicants' invention solves all of these problems, and does so with a simple design that makes storage easy and keeps manufacturing costs to a minimum. The present invention includes all the functionality of a standard syringe (including the ability to depress and pull back on the plunger when withdrawing medicines from a multidose vial) independent of the flush chamber. The design of the syringe takes advantage of basic fluid mechanics to keep the flush and the medicine from contacting each other during use. The present invention advances the state of the art by providing a cost-effective single syringe that both administers medication and flushes the intravenous system. By using a single syringe for both purposes, a clinician need only access the intravenous catheter once, thereby decreasing the rate of error and infection. Additionally, the presence of the saline or other solution in the syringe after injection alerts the clinician of the need to flush the system, thus reducing the chance that the flush would be forgotten. Finally, the extra cost and time associated with a second “flush-only” syringe would be eliminated.

### SUMMARY OF THE INVENTION

The present invention is a two-chambered syringe with an outer barrel having an open end for slidably receiving an inner barrel/first piston. A second piston is slidably movable in the inner barrel/first piston. A latching mechanism locks and unlocks the inner barrel/first piston to the second piston. In the locked configuration, the second piston is prevented from substantially all longitudinal movement relative to the inner barrel/first piston, and in the unlocked configuration, the second piston may move longitudinally within the inner barrel. The invention may be used, for example, to administer a medicine from the outer barrel and then administer a flushing solution from the inner barrel. Thus, the invention may be used as a traditional syringe to withdraw medicine from a bottle, either before or after the administration of a second flushing solution contained in the syringe.

A cost-effective single syringe that both administers medication and flushes the intravenous system is needed to improve the standard of care. It is desirable to allow caregivers to follow their standard syringe filling procedures; to not rely on the fluid in the distal chamber to expel the primary fluid (medicine) from the syringe; to include a physical locking mechanism such that the intermediate fluid cannot be expelled accidentally while depressing the plunger during routine filling; to allow filling of the distal chamber from the proximal end (during manufacturing), which enables complete filling of the distal chamber without trapping any large/non injectable air bubbles; to utilize basic fluid mechanics to keep the two fluids separate when disposed within the syringe; and to not limit the volume of medicine that can be filled into the proximal chamber.

Described herein are syringe devices, systems and methods.

In general, a syringe includes a cartridge and a second chamber. The cartridge includes a first chamber, a second end, and a locking mechanism. The second end is movable within the first chamber between a first position and a second position. The locking mechanism has a locked configuration and an unlocked configuration and prevents movement of the second end within the first chamber while in the locked configuration. The locking mechanism includes: a flexible arm having a first end coupled to the second end of the cartridge and a second free end; a tab coupled to the second free end; and a groove in the inner surface of the chamber configured to receive the tab. The cartridge is moveable within the second chamber.

This and other embodiments may include one or more of the following features. The tab can include a ramped surface such that when the second end is rotated within the inner surface of the cartridge, the ramped surface allows the tab to slide more easily out of the groove. The tab can include two ramped surfaces such that the second end may be rotated in two directions within the first chamber. The syringe can further include an adjacent groove adapted to receive the tab when the locking mechanism is in the unlocked configuration. The tab can be substantially trapezoidal shaped. The tab can be substantially semi-circular shaped. The syringe can further include a second groove configured to receive the tab when the second end is in the second position. The second groove can extend around the circumference of the cartridge. The syringe can further include a first ridge on the inner surface of the cartridge, wherein the ridge is configured to prevent the withdrawal of the second end from the first chamber. The syringe can further include a second ridge on the outer surface of the second end of the cartridge and a second ridge, wherein the first ridge is configured to engage with the second ridge such that withdrawal of the second end from the first chamber is prevented. The cartridge can further include a first end that defines a conduit in liquid communication with the first chamber. The first end of the cartridge can be coupled to the first chamber such that when the second end of the cartridge is rotated within the first chamber, the first end is not rotated. The first chamber can have a noncircular cross section. The syringe can further include indicia that signify when the locking mechanism is in the locked configuration and when the locking mechanism is in an unlocked configuration. The syringe can further include a ridge coupled to an end of the groove, wherein the ridge is configured to prevent the tab from reentering the groove after it has been released. The second end of the cartridge can further include a handle sized and configured to move the second end within the first chamber.

In general, a method of using a syringe having a cartridge having a first chamber, a second end moveable within the first chamber, and a locking mechanism, wherein the syringe further includes a second chamber having an outlet, includes: expelling a liquid from the second chamber through the outlet by moving the cartridge within the second chamber toward the outlet; rotating the second end of the cartridge with respect to the first chamber to release the locking mechanism; and expelling a second liquid from the first chamber through the outlet by moving the second end of the cartridge within the first chamber toward the outlet.

This and other embodiments may include one or more of the following features. The second end can include a tab having a ramp, and rotating can include rotating the tab against a groove defined by the inner wall of the first chamber such that the tab is released from the groove. The rotating step can further include further rotating the tab after the tab is released by the groove such that the tab is received by a second adjacent groove.

### BRIEF DESCRIPTION OF THE DRAWINGS

FIG. 1 is an exploded perspective view of a two-chambered syringe according to an embodiment of the present invention.

FIG. 2 is an exploded perspective view of a two-chambered syringe according to an embodiment of the present invention.

FIG. 3 is a perspective view of the embodiment depicted in FIG. 1.

FIG. 4 is a perspective view of the embodiment depicted in FIG. 2.

FIG. 5 is a side cross-sectional view of the embodiment depicted in FIG. 1, with the inner barrel/first piston full of a liquid such as a saline solution.

FIG. 6 is a side cross-sectional view of the embodiment depicted in FIG. 1, with the second piston partially depressed, thereby expelling some of the liquid.

FIG. 7 is a perspective view of the inner barrel/first piston and sealing ring depicted in FIG. 1.

FIG. 8 is a perspective view of the inner barrel/first piston and sealing ring according to an alternative embodiment of the present invention.

FIGS. 9-14 are perspective views of the proximal end of the second piston and sealing ring according to alternative embodiments of the present invention.

FIGS. 15(a)-(g) are side cross-sectional views of various stages of operation of the two-chambered syringe depicted in FIG. 1. FIG. 15(a) depicts the syringe as delivered to the clinician. FIG. 15(b) depicts the front chamber being filled with air. FIG. 15(c) depicts the air being injected into a medicine bottle. FIG. 15(d) depicts the withdrawal of medicine from a bottle into the front chamber. FIG. 15(e) depicts the administration of the medicine to a patient. FIG. 15(f) depicts unlocking the front chamber from the back chamber. FIG. 15(f) depicts the administration of the prefilled flush solution.

FIG. 16 is a perspective cut away view of the inner barrel/first piston showing the raised track and rear lip.

FIGS. 17(a) and (b) are perspective views of a portion of the inner barrel of a two-chambered syringe according to an embodiment of the present invention.

FIG. 18 is a side cross-sectional view of the embodiment depicted in FIG. 1, with the inner barrel/first piston full of a liquid such that there is a liquid-air interface at or within the conduit.

FIGS. 18A and B represent the balance of forces inside a conduit of a syringe.

FIGS. 18C-F illustrate multiple embodiments of the shape of a conduit of a syringe.

FIG. 19 is a side cross-sectional view of an embodiment of a syringe including an end cap.

FIGS. 20A-20D illustrate multiple embodiments of a method of filling a syringe.

FIGS. 21A-21H illustrate multiple embodiments of the shape of a bubble in the conduit of a syringe.

FIGS. 22A-23 are perspective views of an embodiment of a syringe including a locking mechanism.

FIGS. 24-25 are perspective views of an embodiment of a syringe including a locking mechanism and additional grooves.

FIG. 26A is a perspective view of an embodiment of a syringe including a locking mechanism and a ridge. FIG. 26B is a cross-section of FIG. 26A.

FIGS. 27-28 illustrate various embodiments of a syringe wherein a first end may be fixed with respect to the outer barrel.

FIG. 29 is a perspective view of an embodiment of a syringe including indicia.

FIG. 30 illustrates a groove of a locking mechanism including a ridge.

### DETAILED DESCRIPTION OF THE INVENTION

Described herein are syringe devices, systems and methods. In general, the syringe may include a second chamber and a cartridge movable within the second chamber. The cartridge includes a cartridge chamber (first chamber), a first end that defines a conduit in fluid communication with the cartridge chamber and the second chamber, a liquid disposed within the cartridge chamber such that there is a liquid-air interface at or within the conduit, wherein the liquid has a fluid property such that the liquid-air interface, cooperating with the conduit and the fixed volume of the cartridge, prevents movement of the liquid out of the conduit, a second end, movable within the cartridge chamber, and a locking mechanism having a locked configuration and an unlocked configuration, the locking mechanism preventing movement of the second end within the cartridge chamber while in the locked configuration. In general, the methods of filling a syringe during manufacturing may include the steps of injecting a liquid into a cartridge chamber through a conduit of the cartridge and creating a liquid-air interface within the conduit, wherein the liquid-air interface, cooperating with the conduit, prevents movement of the liquid out of the conduit. In general, the methods of using a syringe may include the steps of drawing a second liquid into the second chamber through the proximal outlet by moving the cartridge distally within the first chamber and creating a second liquid-air interface within the conduit, wherein the second liquid-air interface, cooperating with the conduit and locked prefilled cartridge, prevents movement of the first liquid out of the conduit and prevents movement of the second liquid into the conduit.

The syringe devices, systems, methods, and any combination thereof described herein provide at least the following advantages. First, the syringe described herein does not rely on an intermediate fluid in the distal chamber to expel the primary fluid (medicine) from the syringe. Not using an intermediate fluid to expel the primary fluid avoids mixture that can occur between the two fluids when the syringe is used in standard fashion. Ensuring that the two fluids are not mixed ensures that the patient receives the correct fluids during treatment. As a result, the syringe is more versatile and reliable.

Further, the syringe cartridge includes a conduit designed to keep two fluids in a multi-chamber syringe separate from one another. Keeping the two fluids separate from one another avoids mixing of the two fluids. As noted above, avoiding mixing ensures that the patient receives the correct fluid during treatment, making the syringe more versatile and reliable.

Moreover, prefilled saline flush cannot be inadvertently expelled during routine use. This is an advantage, because, when using a syringe, a caretaker will typically eject air from the proximal chamber prior to drawing a medicine into the proximal chamber, as described later. In pushing the air from the proximal chamber in a prior art syringe lacking a locking mechanism, the caretaker could easily cause the plunger to contact the proximal end of the syringe barrel which would prematurely open the valve, thereby accidentally expelling the flushing liquid. Accidental expelling of the intermediate fluid can cause mixing of the two fluids in the syringe. A physical locking mechanism in conjunction with the other syringe features, as described herein, will therefore keep the fluids from mixing, even in similar stresses and situations. As discussed above, avoiding mixing ensures that the syringe is more versatile and reliable.

Additionally, the syringe described herein provides the advantage that it allows the filling of the distal chamber from the proximal end during manufacturing. Filling the distal chamber from the proximal end during manufacturing enables complete filling of the distal chamber without trapping any large/non injectable air bubbles. Some prior art syringes that are adapted to deliver multiple fluids require filling procedures that include placing an intermediate sliding stopper into a conventional syringe barrel, then filling the distal chamber from the distal end with a liquid, such as saline, and subsequently installing the plunger assembly. By filling the distal chamber with saline before installing the conventional syringe plunger, the prior art syringe has the disadvantage of reliance on the compressibility of the gas trapped in the distal chamber for a successful installation of the plunger. Therefore, by allowing the filling of the distal chamber without trapping large air bubbles, the syringe described herein can provide more accurate and reliable administration. Further, filling the distal chamber during manufacturing gives the caretaker the ability to fill the proximal chamber with a necessary amount of medicine at the time of administration. Some prior art syringes that are adapted to deliver multiple fluids require that they be provided to a caregiver with prefilled distal (saline) and proximal (medicine) chambers. Such a requirement is not desirable, as many patients require different doses of the same medication. If hospitals were to adopt the use of syringes prefilled with medication, it would cause a tremendous storage and utilization problem. Thus, giving the caretaker the ability to fill the proximal chamber with a necessary amount of medicine at the time of administration advantageously allows patients to receive varying amounts of medications.

Additionally, the syringe described herein allows caregivers to follow their standard syringe filling procedures. The most common procedure a clinician uses to fill an empty syringe with medication includes the steps of (1) fitting a syringe with a needle (metal or plastic) to penetrate the seal on a medicine bottle; (2) pulling the handle of the syringe back (distally) to draw air into the syringe of equal or greater volume than the medicine that is to be withdrawn; (3) inserting the air filled syringe with attached needle into the medicine bottle; (4) depressing (pushing proximally) the plunger to inject the air into the medicine bottle; (5) pulling the handle of the syringe back (distally) to draw medicine from the bottle into the syringe; and (6) withdrawing the needle/syringe from the medicine bottle and removing the needle from the syringe. Prior syringes that are adapted to deliver multiple fluids cannot be used in this procedure for at least the reason that during Step 4, after injecting all the air from the proximal chamber of the syringe into the medicine bottle, the plunger will often collide with the internal surface of the inside of the syringe barrel. This collision causes the displaceable valved stopper to open and remain open. Once the valve is open, pulling back on the plunger would cause medicine to flow through the open valve and mix with the contents of distal chamber. Alternatively, if the forward force were continually applied, after the valve was opened, the contents of the distal chamber would flow through the open valve into the medicine bottle. Neither one of these scenarios is acceptable. The syringe described herein, including a physical locking mechanism and separate cartridge (including an inner barrel) which is adapted to use fluid mechanics to keep fluids separate, is ideally suited for a caregiver's standard filling procedure.

A further advantage of the syringe described herein is that it does not limit the volume of medicine that can be filled into the proximal chamber. A disadvantage of some prior syringes that are adapted to deliver multiple fluids by sectioning a standard syringe into two compartments is that the volume of medicine that can be filled into the proximal chamber is limited by the presence of the distal chamber. In general the greater the diameter of the syringe barrel, the less exact a measurement of volume can be made by reading the fluid meniscus against gradations marked on the outside of the syringe. The accuracy required is generally related the total volume of medicine to be administered, the smaller the dose of medicine the more accurate measurement is needed. To solve this problem clinicians use a wide range of syringe sizes depending on the amount of medication to be administered. Syringes from 1 ml to 60 ml are the most commonly used sizes. In the prior art syringes that are adapted to deliver multiple fluids the distal chamber defined by the sliding stopper takes up space within the standard syringe barrel (the effective volume for medication is decreased by the distal chamber volume by about a factor of 2 for a given syringe size) and therefore clinicians would have to use a relatively larger syringe barrel size and therefore less accurate measurements to attempt to administer the same volume of medicine. The syringe described herein includes a separate cartridge that includes the distal chamber, and therefore does not negatively impact the potential size of the proximal chamber and its capability to hold a volume of medicine.

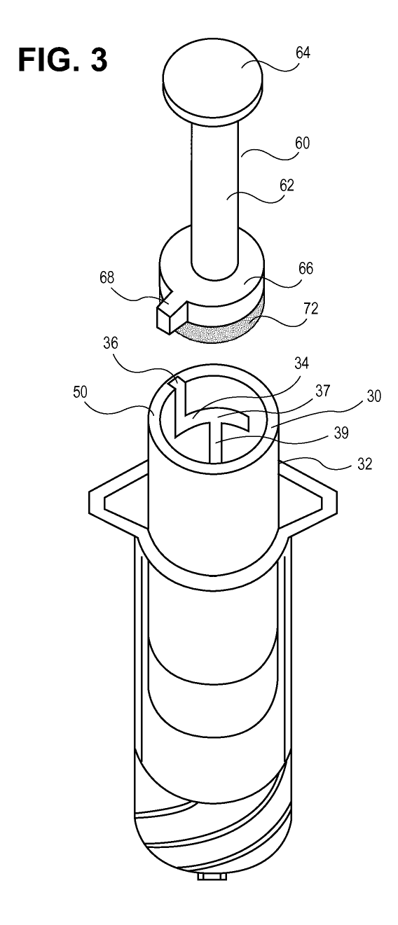

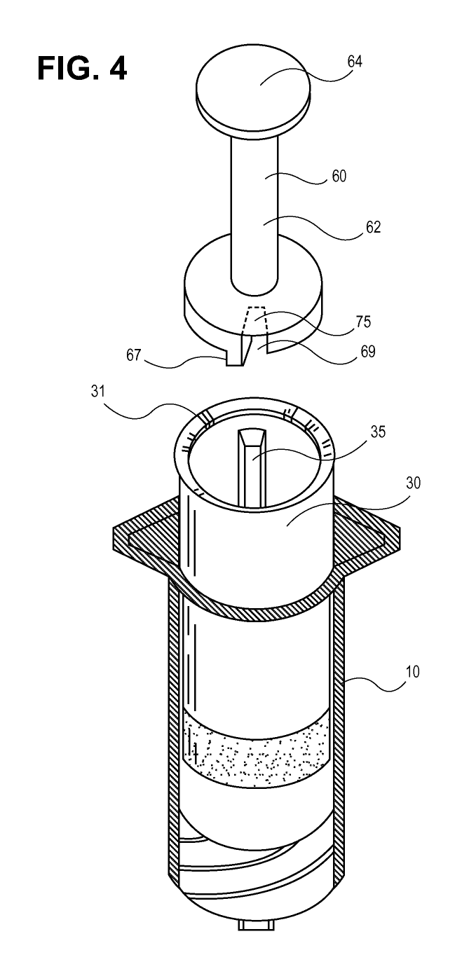

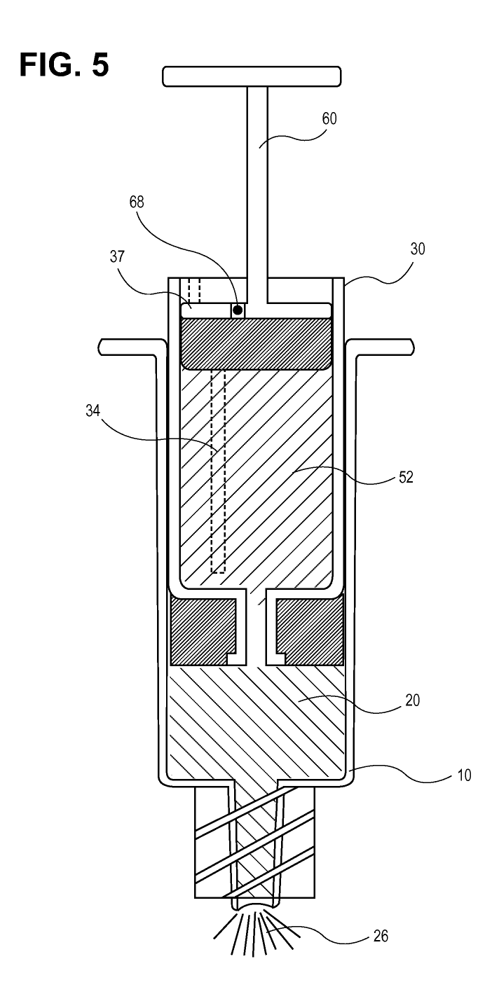

The present invention is a two-chambered syringe with three basic components: (i) an outer barrel 10 for holding a liquid 20, (ii) an inner barrel/first piston 30 for holding a flushing liquid 52, and (iii) a second piston 60. See FIG. 5. The syringe also includes a latching mechanism for controlling the movement of the second piston 60 in the inner barrel/first piston 30. See FIGS. 3 and 4.

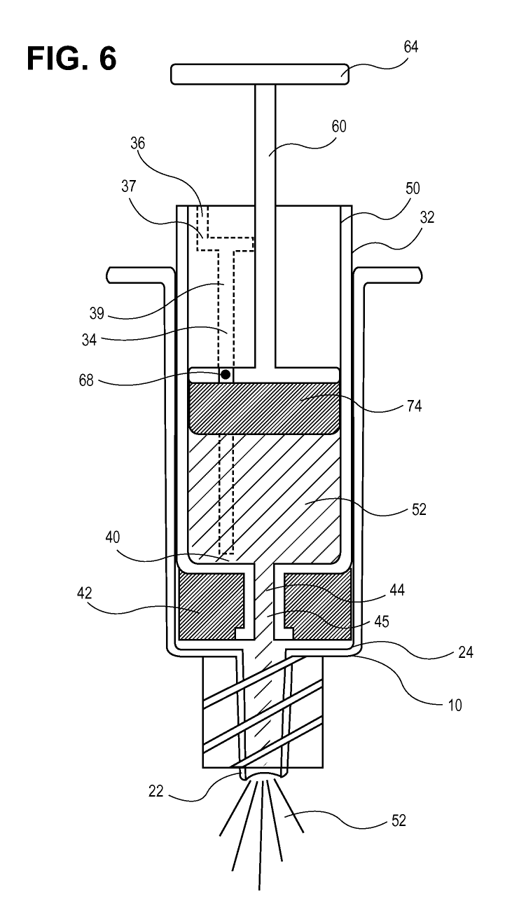

The barrels and pistons may be constructed of polypropylene or other similar inert, nonreactive semi-flexible material. Both barrels 10, 30 are generally circular cylinders. The inner barrel/first piston 30 acts as both a barrel and a piston. That is, it both holds liquid like a barrel, and may be used as a plunger to expel liquid from the outer barrel 10. See FIGS. 5 and 6.

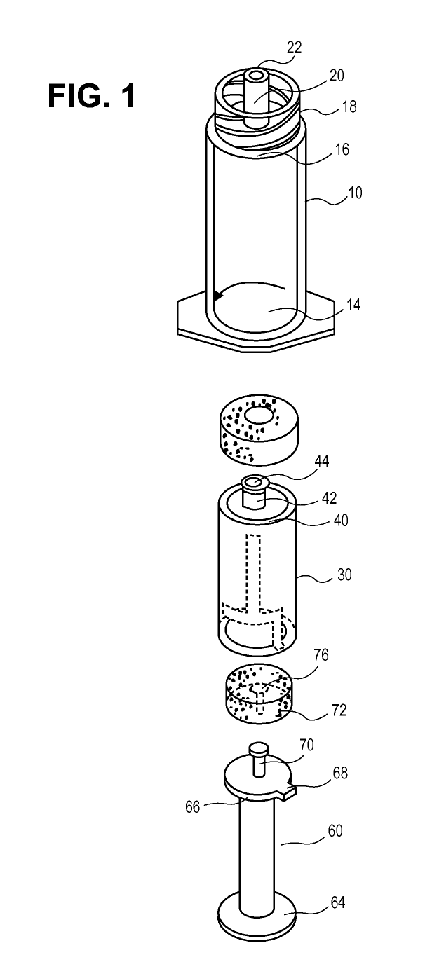

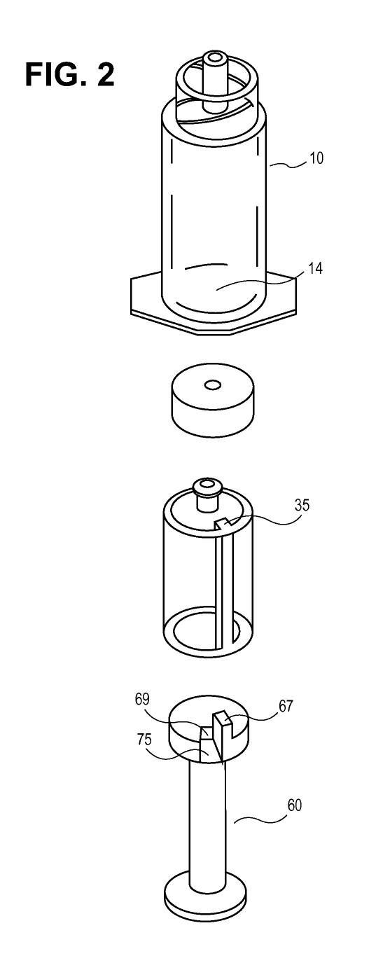

For purposes of this patent, the proximal end of the syringe is the end typically comprising a first conduit 20, while the distal end is the end of the syringe typically comprising the second piston 60 and a gripping handle 64. See FIGS. 1 and 2.

The outer barrel 10 has an outer barrel distal open end 14 adapted for receiving the inner barrel/first piston 30. See FIG. 1. The inner barrel/first piston 30 is slidably contained in outer barrel 10 in a liquid-tight relation, similar to the piston or plunger in syringes common to the art. See FIGS. 1-6 and 15.

In one embodiment, a proximal end 16 of the outer barrel 10 may comprise an adapter 18, such as a luer connector device as disclosed in U.S. Pat. No. 4,452,473 or other locking means common in the art. See FIG. 1. The adapter 18 allows a connection between the present invention and an intravenous system. An outer barrel open proximal end 22 is at the proximal end 16 of the outer barrel 10 and may contain a first conduit 20. See FIG. 1. The distal end of first conduit 20 is in communication with the proximal end 16 of the outer barrel 10, providing a passageway for fluid from either the outer barrel 10 or the inner barrel/first piston 30. See FIG. 1.

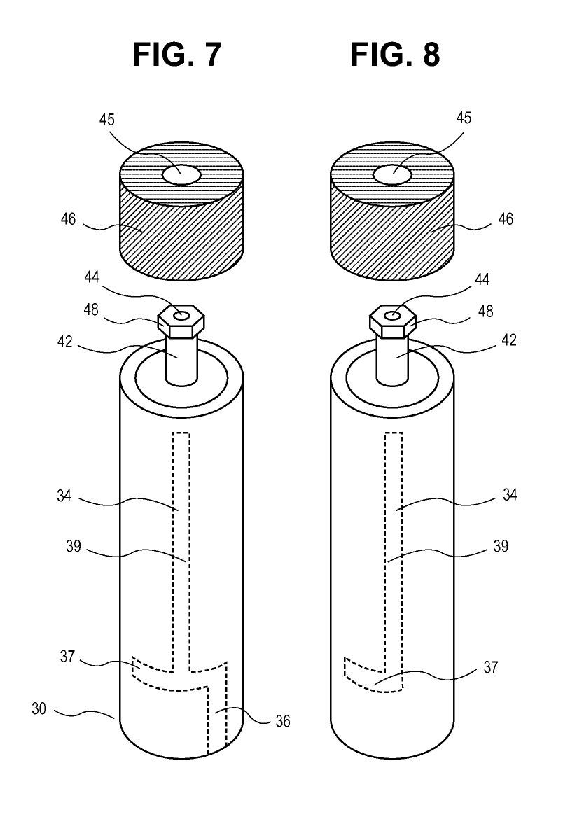

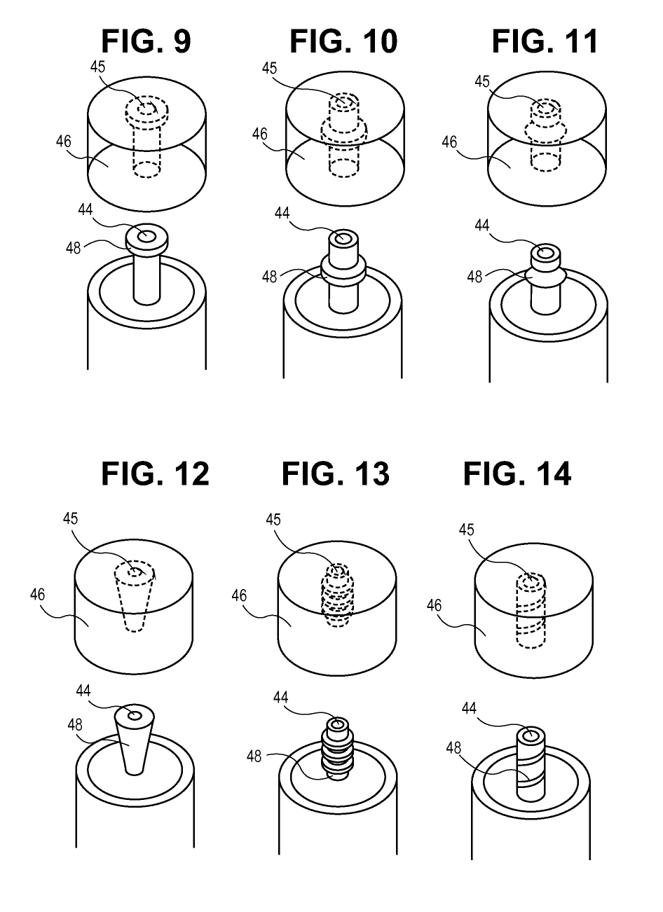

The inner barrel/first piston 30 has an inner barrel/first piston proximal end 40 slidably received within the outer barrel open distal end 14. See FIG. 1. It also includes a hollow projection 42 that extends proximally out of the inner barrel/first piston 30. See FIGS. 1, 9-14. The hollow projection 42 defines a second conduit 44 through which liquid flows from the inner barrel/first piston 30 to the outer barrel 10. See FIGS. 1 and 6-8. The hollow projection 42 has a flared tip 48 that secures a first sealing ring 46, as shown in FIGS. 7 and 8. The flared tip 48 may take many different forms, as shown in FIGS. 9-14.

The first sealing ring 46 comprises a sealing ring conduit 45 through which extends the hollow projection 42. See FIGS. 7-14. The first sealing ring 46 is substantially the same diameter as both the inner barrel/first piston outer wall 32 and the outer barrel inner wall 24, creating a liquid tight seal between the inner barrel/first piston 30 and the outer barrel 10. See FIG. 6. Thus, the only fluid connection between the inner barrel/first piston 30 and the outer barrel 10 is through the second conduit 44 and the sealing ring conduit 45. The sealing ring 46 may be constructed of an elastic material such as natural or synthetic rubber.

The flushing liquid 52 is inside the inner barrel/first piston 30. See FIGS. 5, 5-6. The flushing liquid 52 may be a saline solution, or any other suitable solution, such as heparin, when anticoagulation is desired, or antibiotics, when a line infection is being treated.

The flushing liquid 52 occupies substantially all of the space defined by the inner barrel/first piston inner wall 50, and initially extends partially through the second conduit 44 defined by the hollow projection 42. See FIG. 6. Because the flushing liquid 52 only extends partially through the second conduit 44, the flushing liquid 52 remains isolated from any liquid later drawn into the outer barrel 10.

The second piston 60 is slidably placed within the inner barrel/first piston 30. See FIGS. 3-5 and 15. The second piston 60 comprises a second piston proximal end 66 further comprising a solid projection 70 that fits through an aperture 76 in a second sealing ring 72, thereby attaching the second piston 60 to the second sealing ring 72. See FIGS. 1, 3. The second sealing ring 72 is of substantially equal diameter to the inner barrel/first piston inner wall 50, and is created from an elastic rubber-like material that provides a liquid-tight seal for the inner barrel/first piston 30. See FIG. 3. Alternatively, this liquid-tight seal may be created by a similar rubber-like sealing material 61 placed around the periphery of the proximal end of the second piston 60. See FIG. 17. The second piston 60 moves in and out of the lumen of inner barrel/first piston 30, thereby dispensing liquid from or drawing liquid into the inner barrel/first piston 30. See FIG. 3.

Extending distally from second piston proximal end 66 is a piston rod 62. See FIGS. 3 and 4. A gripping handle 64 is placed at the most distal end of the second piston 60.

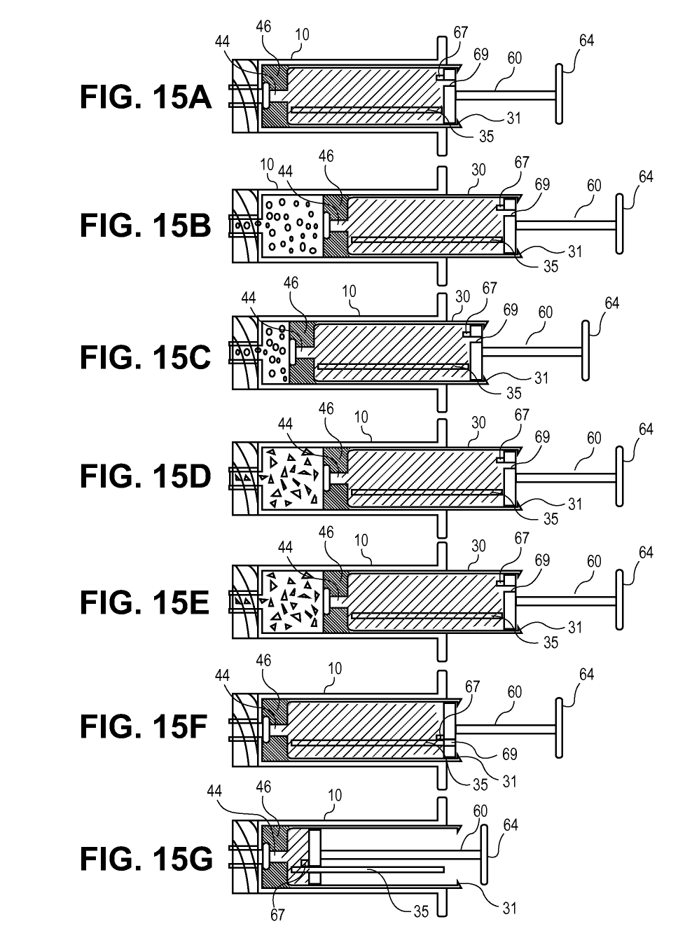

The two-chambered syringe further comprises a latching mechanism that can alternate between an unlocked configuration and a locked configuration. See generally FIGS. 3-4 and 7-8. In the locked configuration, the second piston 60 is longitudinally locked relative to the inner barrel/first piston 30. See FIG. 15(b). In this configuration, the second piston 60 will not move longitudinally relative to the inner barrel/first piston 30. See FIGS. 5 and 15(a)-15(e). However, a longitudinal force applied to the second piston 60 will be transferred proximally and the inner barrel/first piston 30 will move relative to the outer barrel 10.

In the unlocked configuration, the second piston 60 is free to move longitudinally relative to the inner barrel/first piston 30. See FIGS. 6 and 15(f)-15(g). Thus, the contents of the inner barrel/first piston 30 are ejected through the second conduit 44 when the second piston 60 is depressed. When the second piston 60 is retracted, the inner barrel/first piston 30 will provide sufficient suction to draw in the contents of the outer barrel 10 through the second conduit 44.

In one embodiment, the latching mechanism comprises a projection 68, extending outward radially from near the second piston proximal end 66. See FIGS. 1 and 3. In this embodiment, the projection is constructed of a polypropylene or other similar inert, nonreactive semi-flexible material the same as or similar to that comprising the barrels and pistons of the syringe. While the radial width of the projection 68 shown in FIGS. 1 and 3 is small relative to the distance around piston rod 62, the same principle preventing movement of the piston rod 62 would apply regardless of the radial width or shape of projection 68. See FIG. 3.

This projection fits snugly into a groove 34 cut into the inner barrel/first piston inner wall 50, thereby allowing the second piston 60 to only move according a path of movement defined by groove 34. See FIGS. 3 and 6.

The groove 34 includes a longitudinal portion 39 extending longitudinally along the inner barrel/first piston inner wall 50, ending at the inner barrel/first piston proximal end 40. See FIG. 6. Near the distal end of the inner barrel/first piston 30, the longitudinal portion 39 makes a substantially right angle and continues circumferentially around the inner barrel/first piston inner wall 50 as a radial portion 37. See FIGS. 3, 6, and 8. In one embodiment, the radial portion 37 of the groove 34 extends less than one half of one revolution of the perimeter around the inner barrel/first piston inner wall 50. See FIGS. 7 and 8.

In one embodiment, the groove 34 continues to substantially the distal end of inner barrel/first piston 30, outlining a track ultimately leading to a projection entry point 36. See FIGS. 3, 6, 7. The projection entry point 36 serves as an entrance to the groove 34 for the projection 68, simplifying the assembly process for the syringe and reducing the cost of construction. In the alternate embodiment, shown in FIG. 8, the second piston 60 with protrusion 68 would be installed into the inner barrel by applying sufficient pressure to temporarily flex the plastic allowing a press-fit construction. See FIGS. 3 and 8.

When the second piston 60 is in the fully extended position, the projection 68 will lie in the radial portion 37 of the groove 34. See FIG. 5. From this position, the second piston 60 may be axially rotated, and the projection 68 will slide along the radial portion 37 of the groove 34. Additionally, the second piston 60 and the inner barrel/first piston 30 are longitudinally locked together, and in this fixed position the two components function collectively as one piston relative to the outer barrel 10. See FIGS. 5 and 15(a)-15(e). The syringe may then be used in the same manner as a conventional one-chambered syringe, as described later herein.

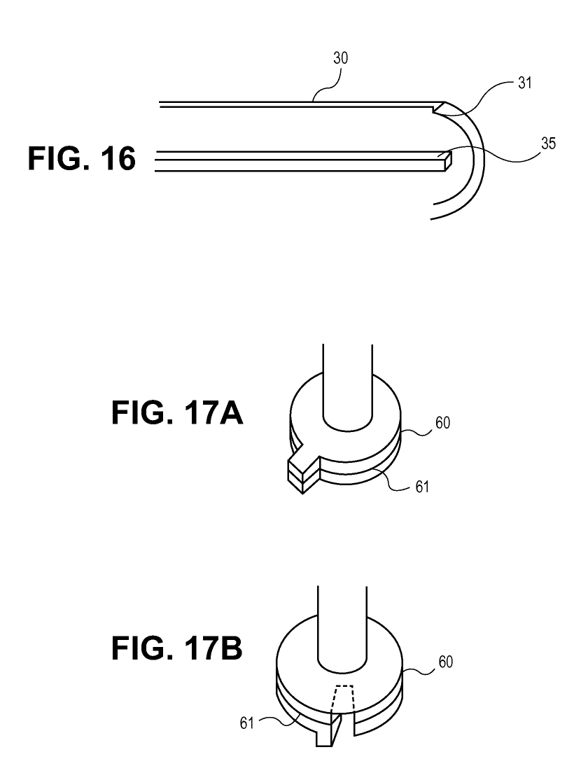

In yet another embodiment, instead of comprising a track defined by an indented groove on the inner barrel/first piston 30, the syringe comprises a track defined by a raised track outlining the same path previously defined by the groove 34. See FIGS. 2-4, and 16. Correspondingly, the second piston 60 comprises an indentation 69 instead of the projection 68. See FIGS. 2-4. In this configuration raised track 35 fits snugly into indentation 69, thus defining a track for the second piston 60 to follow when in the unlocked position. See FIGS. 2 and 4. In this embodiment, the track need not extend longitudinally the entire length of the inner barrel to accomplish the locking feature.

To ensure the saline does not leak backwards out of the flush chamber, the second piston 60 may additionally comprise breakaway guard 75, which provides a cover over the indentation 69. The breakaway guard 75 may be a layer of plastic that is capable of being punctured by raised track 35 when the operator applies sufficient force. The operator of the syringe will feel the resistance and subsequent release as the breakaway guard is punctured. See FIGS. 2, 4, and 16. The need for this guard may be circumvented by making a rear lip 31 large enough to prevent backward flow of the flush solution. The lip 31 of the inner barrel enables a unidirectional press fit construction (due to the sloped angle of the lip 31) in which the second plunger may be easily slid into the inner barrel, but cannot be easily removed. Thus, the second piston 60 is effectively trapped between the raised track 35 and the lip 31 thus preventing the second piston from moving longitudinally with respect to the inner barrel/first piston when the second piston is in the locked configuration. See FIGS. 15(a)-15(f).

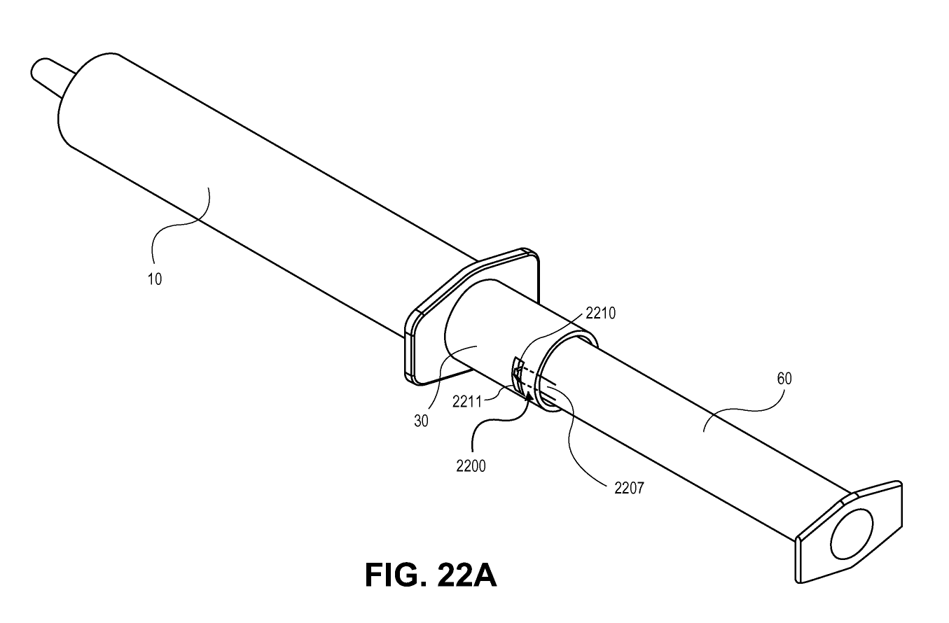

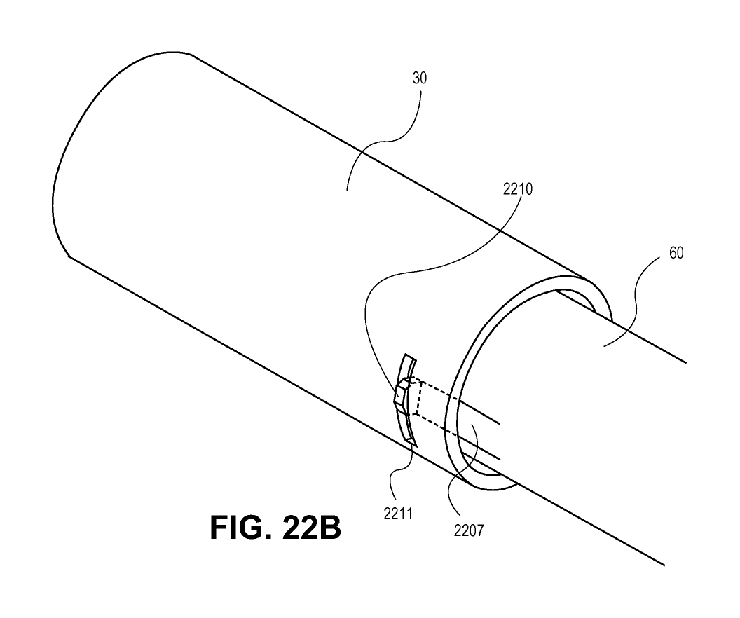

Other latching mechanisms may be used, some of which are described further below with respect to FIGS. 22-30. For purposes of this patent, “latching mechanism” refers generically to any structure that can lock and unlock the inner barrel/first piston 30 relative to the second piston 60. See FIG. 1.

One advantage of applicant's device is that the syringe may function as a traditional syringe, independent of the internal flush chamber in the inner barrel/first piston 30. See FIGS. 15(b)-15(e). Additionally, this syringe may be used to dispense a flush solution without filling the outer chamber with a second liquid or gas.

In operation, the syringe will typically first be in the locked position so medicine withdrawn from a bottle fills the outer chamber 10. See FIG. 15(a)-(d). When medication is administered directly to a vein, a clinician using a traditional syringe will often confirm that a vein has been pierced by drawing a small amount of blood into the syringe, prior to injection of the medication. This device allows for this normal operation to be performed when the device is in the locked configuration. See FIGS. 15(b)-15(c).

Because the flushing liquid 52 does not extend through the second conduit 44, it will not mix with fluid drawn into the outer chamber 10. In a separate embodiment (shown in FIG. 5), flushing liquid 52 extends only partially through the second conduit 44, but not enough to mix with fluid drawn into outer chamber 10. The two fluids will not come in contact with each other due to basic fluid mechanics. That is, surface tension of the fluid drawn into the outer chamber 10 prevents it from entering the second conduit 44. The flushing liquid 52 does not move through the second conduit because as it completely fills the inner barrel/first piston 30, the negative pressure created inside the outer barrel 10 when fluid is drawn in, is not great enough to displace the flushing liquid 52 from the inner barrel/first piston 30.

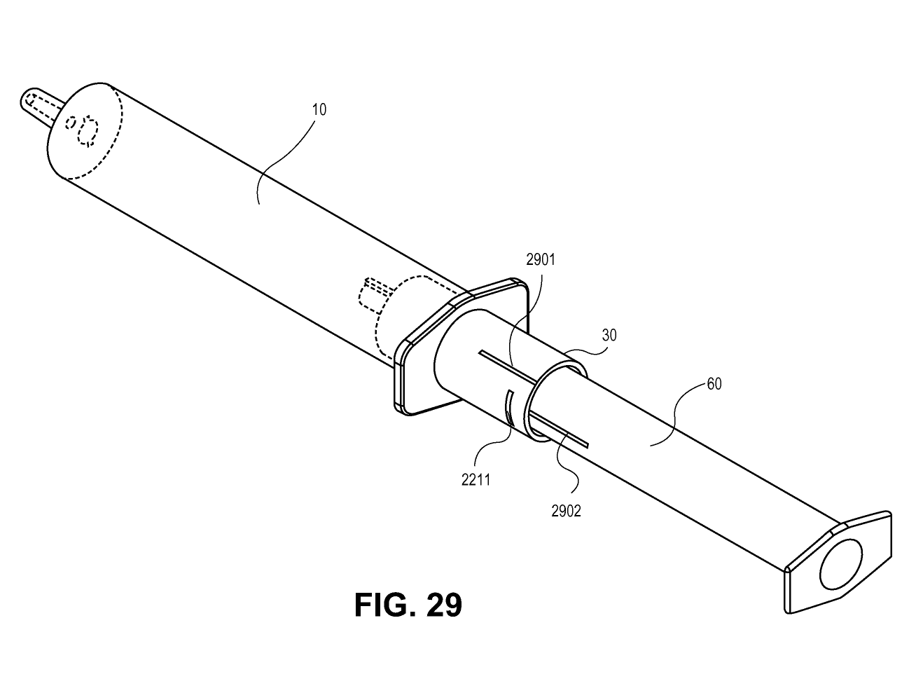

Next, while the syringe is still in the locked configuration, the contents of the outer barrel 10 may be delivered to a patient by depressing the second piston 60. See FIGS. 15(e)-15(f). After injecting the medication, the operator may axially rotate the second piston 60 until the longitudinal portion 39 of either the groove 34 or the track 35 defines the path of movement. See FIGS. 6 and 15(f)-15(g). In the embodiments shown in FIG. 2 and FIG. 8 the clinician may confirm this alignment upon feeling that the axial rotation is halted by forward projection 67. In the embodiments shown in FIG. 1 and FIG. 3, a clinician may confirm this alignment by rotating the second piston 60 until an indicating mark on second piston 60 is longitudinally in line with a mark on the inner barrel/first piston 30 or the outer barrel 10, as described below with respect to FIG. 29. From this position, the second piston 60 may be longitudinally moved down the length of the inner barrel/first piston 30, thereby emptying the contents of the inner barrel/first piston 30 into the outer barrel 10 and then into the catheter. See FIGS. 6 and 15(f)-15(g).

In the embodiment shown in FIG. 2, after the outer barrel is dispensed the second plunger may be rotated axially until the forward protrusion 67 meets the raised track 35, impeding further rotation. From this position, proper alignment of the track and indentation is assured because the forward protrusion 67 is adjacent to the indentation 69. Next, the operator would depress the second piston 60 a second time, emptying the contents of the inner barrel through second conduit 44. See FIGS. 6 and 15(f)-15(g). Preferably, at this point in the process, the medication from the outer barrel 10 is already expelled into the intravenous system, and thus the contents of the inner barrel/first piston 30 may be used to flush any remaining medication into the patient.

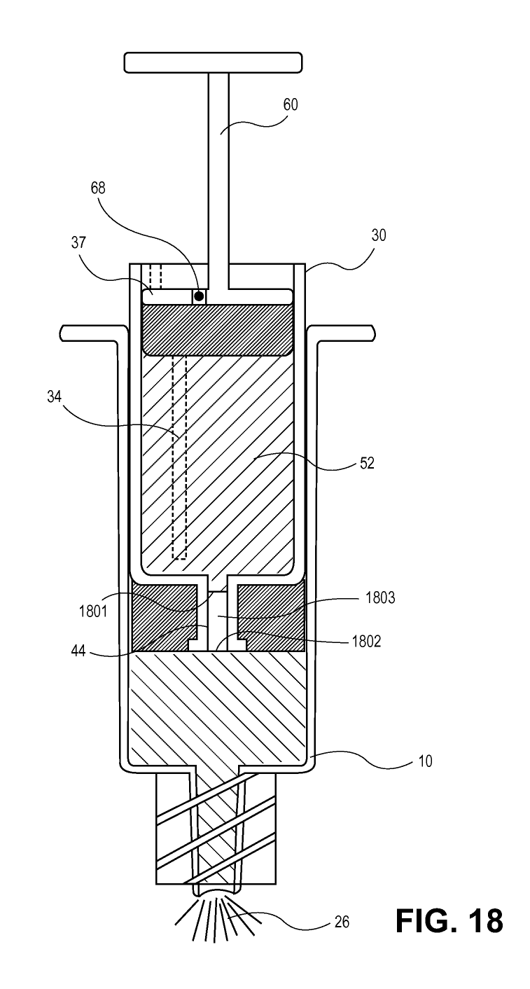

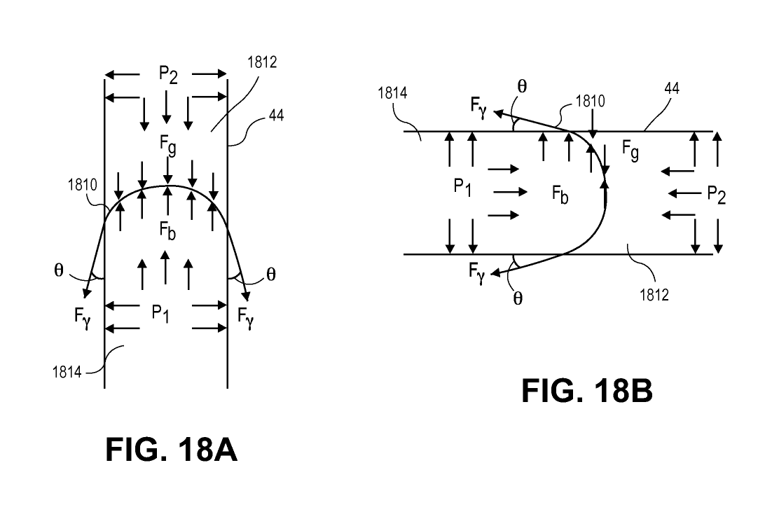

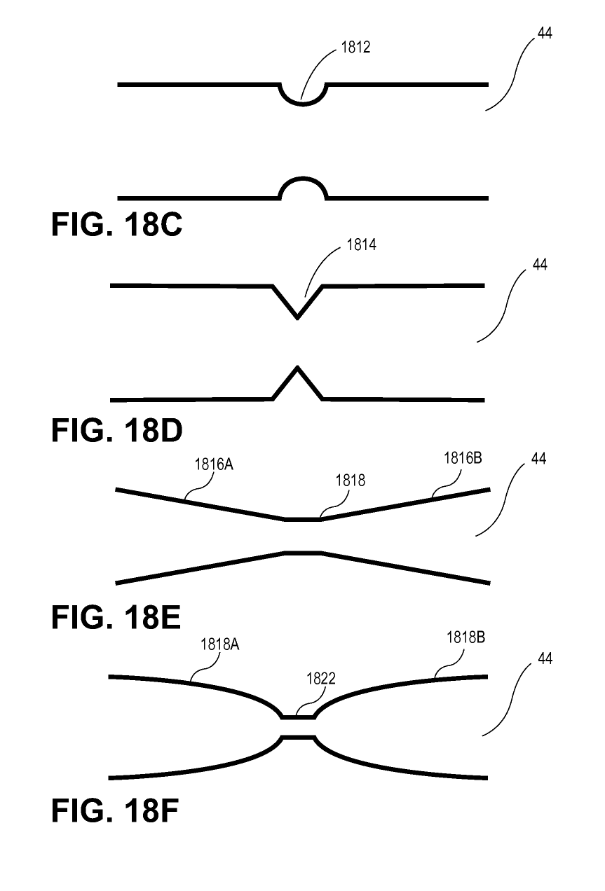

As shown in FIG. 18, and as described above, the syringe described herein includes an outer barrel 10 and an inner barrel 30 that is movable within the outer barrel 10. The inner barrel 30 has a first end which defines a conduit 44 that is in fluid communication with the outer barrel 10. The inner barrel 30 is movable within the outer barrel 10 such that the inner barrel 30 can act as a piston. A second piston 60 can be located within the inner barrel 30 so as to draw or flush fluid in or out of the inner barrel 30. The inner barrel 30, piston 60, and conduit 44 can together be called a “cartridge” of the syringe. The syringe may further include a locking mechanism on the inner barrel 30 (e.g., having groove 34 as shown in FIG. 18). The locking mechanism prevents movement of the piston 60 within the inner barrel 30 while in the locked configuration. The outer barrel 10 defines a proximal chamber and the inner barrel 30 defines a distal chamber. The proximal chamber will typically hold a medicine, while the distal chamber will typically hold a prefilled flushing liquid such as saline.

The inner barrel 30 is thus adapted to hold a liquid 52 in the distal chamber. A liquid-air interface 1801 is created within the conduit 44, which, along with the fixed volume created by the locking mechanism, prevents movement of the liquid out of the conduit 44. Further, the liquid 52 and the liquid of the outer barrel 10 will not come in contact with each other due to basic fluid mechanics. That is, the physical properties of the conduit 44 and the surface tension of the fluid drawn into the outer barrel 10 prevent the fluid from entering the second conduit 44, while the physical properties of the conduit 44 and the surface tension of the liquid 52 within the inner barrel 30 prevent the liquid 52 from exiting the inner barrel 30 through the conduit 44. Moreover, the flushing liquid 52 does not move through the conduit 44 because it completely fills the inner barrel 30, which is locked with a fixed volume. As a result, the negative pressure created inside the outer barrel 10 when fluid is drawn in is not great enough to displace the liquid 52 from the inner barrel.

After a clinician fills the outer barrel 10 with a fluid or medicine, larger air bubbles in the inner barrel 10 are removed by a standard process of tapping the side of the syringe to cause the air bubbles to coalesce into one large bubble which is expelled by orienting the syringe such that the air bubble is near the syringe tip and then the plunger is depressed to expel this air. However, the air that was originally in the conduit 44 as a result of filling of the inner barrel 30 remains in place due to the force of the surface tension and the fixed volume of the inner barrel 30. The standard process of removing the larger air bubbles does not dislodge the retained air within the conduit. When the outer barrel 10 is filled with a fluid, the distal end of the proximal chamber includes a second liquid-air interface 1802, forming a bubble or air pocket 1803 that plugs the conduit 44. The gas-filled space defined by the inner wall of the conduit 44 and the two fluid interfaces act as a plug to keep the two fluids separate until the inner barrel 30 is unlocked.

Because the liquid 52 does not extend through or extends only partially through the second conduit 44, it will not mix with fluid drawn into the outer chamber 10. That is, the bubble 1803 acts as a barrier between the fluids in the two chambers. The bubble is preferably small, having a volume of about 0.01 ml to 0.1 ml. In some embodiments, the volume is about 0.024 ml. This bubble is of substantially similar size to, or smaller than, micro bubbles which routinely form in conventional syringes used to administer medicine to patients. In fact, most conventional prefilled saline syringes contain incidental gas bubbles larger than about 0.024 ml. Clinically, small bubbles are unavoidable and completely harmless. Once injected into the patient they are broken up in the capillary bed and absorbed from the circulation without any effects to the patient. A large bubble is commonly defined as a bubble having a volume greater than 50 ml. A large bubble can behave differently from a small bubble and can be dangerous to a patient if injected into their blood stream. In contrast, the small bubble formed in the conduit 44 is not dangerous to a patient and can be beneficial for creating the valve feature, as described herein.

The bubble 1803 is held into position by forces that include surface tension, buoyancy, gravity, resistance to flow, the shape of the conduit, and the fixed volume of the cartridge chamber. Thus, the force required to dislodge the bubble depends on the dimensions of the conduit 44, the fluid viscosity and the compliance of the saline chamber. It also depends on the surface tension, contact angle of the fluid, wettability of the surface, and shape of the conduit 44. FIGS. 18A and 18B represent the balance of forces in the conduit 44 when a liquid-air interface 1810 is formed between air 1812 (e.g. the bubble 1803) and liquid 1814. The buoyancy force, Fb, is directed upward with respect to the Earth's surface. The surface tension, Fγ, is directed along the contact angle at the edge of the liquid-air interface 1810. The gravity force, Fg, is directed downward with respect to the Earth's surface. The pressure in the fluid, P1, is applied equally and perpendicular to all surfaces, including the liquid-air interface 1810 and the inner walls of the conduit 44. Likewise, the pressure in the air, P2, is applied equally to all surfaces, including the liquid-air interface 1810 and the inner walls of the conduit 44.

The syringe system is nearly rigid; the volume contained within the inner barrel 30 of the syringe is constant if the second piston 60 is fixed in position by the locking mechanism. As a result, the bubble 1803 in the conduit 44 will remain in position. When the locking mechanism is unlocked, however, and the second piston 60 is depressed, the bubble's rear surface is disrupted by the forward flow of liquid 52, causing the bubble to be propelled forward into the outer chamber 10 and/or out through the proximal outlet of the outer chamber 10.

The stability of the bubble position is related to the force of attachment to the wall of the conduit 44. This can be measured as a pressure needed to detach and move the bubble. The pressure needed to move the bubble out of the conduit is a function of the following variables: dimensions of the conduit 44, the fluid viscosity, the compliance of the inner barrel 30, the surface tension, contact angle of the fluid and wettability of the surface. These dependencies are detailed below.

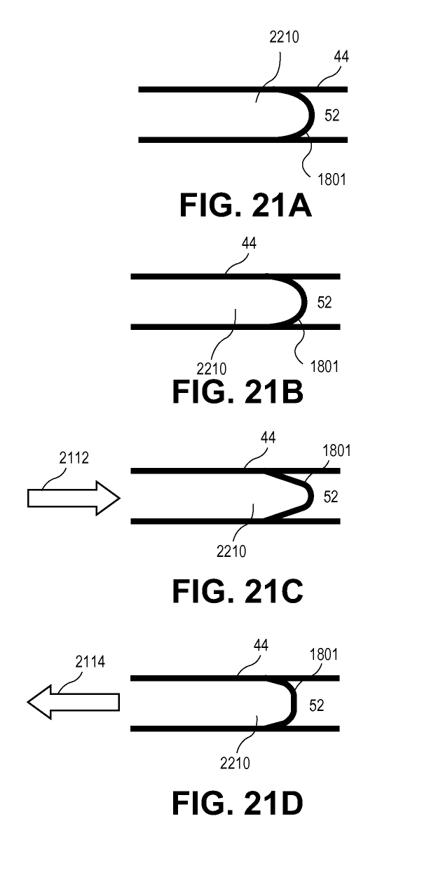

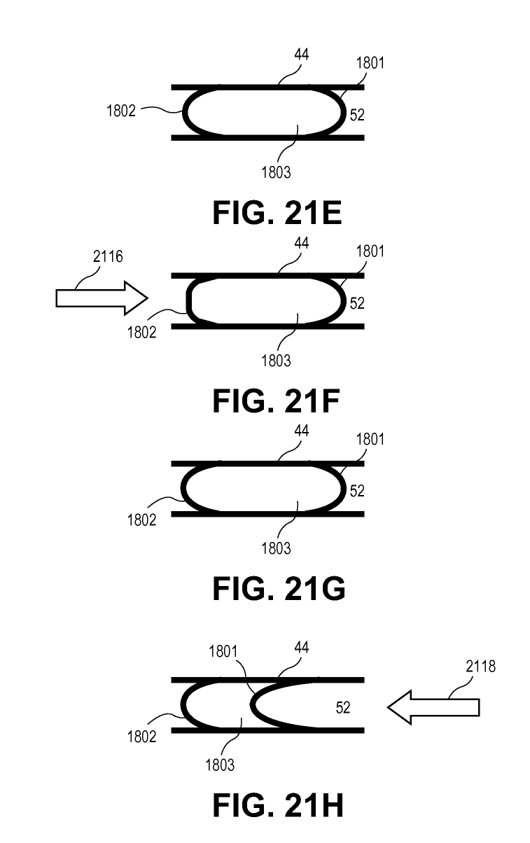

In some embodiments, the conduit 44 between the proximal and distal chambers has a cylindrical shape, with radius R and length L. When the volume of the bubble is greater than that of a sphere equal to 4/3πR³, the bubble elongates in the conduit into a cigar shape, as shown in FIGS. 18 and 21E. The external force holding the bubble stationary depends on the bubble half length, H and the bubble radius, R in the following way:

F∝H²R

This equation assumes that

$$\frac{{H}^{2}}{{R}^{2}} > 1$$

Thus, the length of the bubble 2H is maximum when 2H is equal to L, and the resistive force of the bubble will increase more when length is increased than when radius is increased. Accordingly, the dimensions of the conduit can be chosen such that the bubble keeps the fluid in the first chamber and the fluid in the second chamber apart. In one particular embodiment, the conduit has a diameter of about 0.069 inches. In one particular embodiment, the conduit has a length of about 0.4 inches.

Viscosity is a fluid property that describes it's resistance to flow. It is also known as the ‘thicknesses’ of the fluid. A higher force will be required to attain the same fluid velocity for a higher viscosity fluid. The resistance of the bubble 1803 is slightly increased with increased viscosity. Most medicines will not have a viscosity difference from saline of a magnitude that would significantly affect the resistance.

The position of the bubble 1803 will also be a function of the compliance of the distal chamber (of inner barrel 30), which can be affected by the relative displacement of the locking mechanism while in the locked configuration. If the locking mechanism is not designed or built with the appropriate rigidity, excess motion (wiggle of the locking mechanism) is capable of producing a change in volume of the distal chamber, this can cause the bubble to be dislodged from the conduit 44. If the bubble can be dislodged from the conduit 44, then a small amount of mixing of the saline with the medicine may occur. The maximum displacement can be determined by the following equation:

$$maximum displacement = \frac{R}{{R}_{distal}} L$$

where Rₚᵣₒₓᵢₘₐₗ is the radius of the proximal chamber and R, L as defined earlier.

The compliance of the proximal chamber (of inner barrel 30) will also be influenced by the compliance of the second end of the cartridge, for example by the compliance of a rubber plunger, C_{plunger} if the rubber plunger comprises part of the conduit. If the rubber plunger compresses significantly under pressure, it can reduce the proximal chamber volume and dislodge the bubble. The compressive volume change over the expected range of pressure should be less than an amount equal to the current conduit 44 volume.

ΔV=ΔP·C_{plunger}<πR²L.

The surface tension of a fluid is a measure of how readily the fluid surface is attracted to another surface. It is a property of a fluid that is related to the surface free energy, and affects the contact angle. The force or pressure needed to dislodge the bubble 1803 from the conduit 44 is increased with increasing surface tension.

Contact angle is classically measured by placing a drop on a horizontal surface and measuring the angle of the drop edge. The contact angle is determined from the position of the interfaces between solid, liquid and gas at equilibrium. If a droplet of water spreads on a solid surface, the contact angle is very small and the surface is considered hydrophilic. If the droplet rounds up, the contact angle is greater than 90°, and the surface is hydrophobic. The contact angle of the fluid used is preferably less than 90° to maintain the bubble seal between the two chambers.

The wettability of a surface is directly related to the contact angle, and is another indication of the balance of forces within the liquid that are cohesive, and those between the liquid and the surface that are adhesive. A hydrophilic contact angle is indicative of a strong attraction between the fluid and the surface, a surface that is considered wetting.

Although the conduit 44 is described above as being cylindrical, the shape of the conduit 44 can be varied to optimize the ability of the conduit 44 to keep air in the conduit 44, and thus to keep the liquids in the proximal and distal chamber separate. For example, as shown in FIG. 18C, the conduit can be cylindrical and include rounded indentations 1812 into the inner circumference of the conduit 44. As shown in FIG. 18D, the conduit 44 can be cylindrical and include sharp indentations 1814 into the inner circumference of the conduit 44. As shown in FIG. 18E, the conduit 44 can include two opposing straight-walled conical portions 1816a, 1816b. A straight cylindrical portion 1818 can be located between the two opposing conical portions 1816a, 1816b. As shown in FIG. 18F, the conduit 44 can include two opposing curved-walled conical portions 1820a, 1820b. A straight cylindrical portion 1822 can be located between the two opposing conical portions 1820a, 1820d.

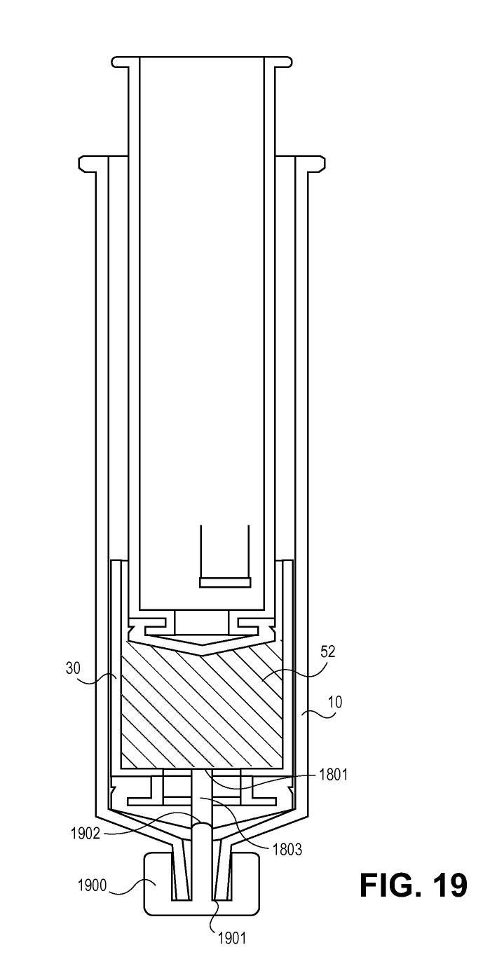

In some embodiments, as shown in FIG. 19, the syringe further includes a removable end cap 1900 coupled to the outlet 1901 of the outer barrel 10 and to the conduit or conduit 44 of the cartridge. As shown, the end cap includes an end surface 1902 within the conduit 44. The end surface 1902, cooperating with the conduit 44, prevents movement of the liquid 52 out of the conduit 44 and out of the inner barrel 30. As shown, the syringe includes a gas bubble 1803 disposed in the conduit 44 between the end surface 1902 of the end cap and the liquid-air interface 1801. In some embodiments, the syringe is designed to be filled with saline by the manufacturer, capped with an end cap 1900 and shipped to the customer. The customer will then remove the cap, and fill the proximal chamber of the outer barrel 10 with medicine as desired.

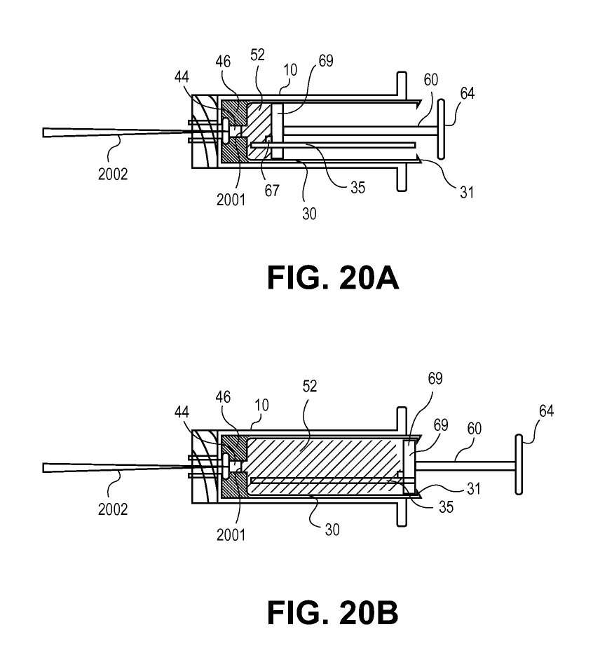

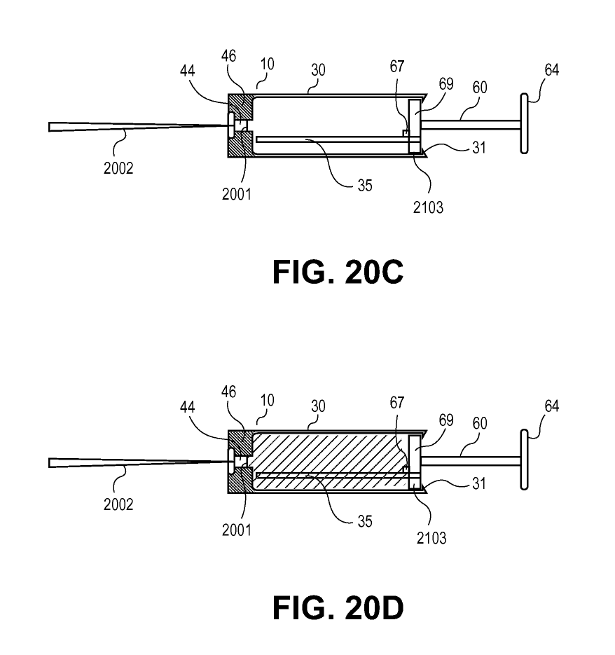

As described above, in some embodiments, the syringe is designed to be filled with a flushing liquid, such as saline, by the manufacturer of the syringe. In general, as shown in FIGS. 20A and 20B or 20C and 20D, a method of filling a syringe cartridge includes the steps of injecting a liquid 52 into the inner barrel 30 through conduit 44 and creating a liquid-air interface 2001 within the conduit 44. The liquid-air interface is maintained during packaging and shipping. The inner barrel chamber is filled up to the distal end of the conduit 44 or beyond, such that the inner barrel 30 is completely filled with fluid and a small volume of air remains in the conduit 44.

The liquid 52 may be injected into the cartridge chamber via a needle or nozzle 2002 positioned within the outlet of the outer barrel 10 and the conduit 44 of the cartridge. In some embodiments, the inner barrel 30 can hold about 1 to 10 ml of liquid, such as 2 to 3 ml of liquid. Ideally the inner barrel 30 holds the smallest volume of fluid, such as saline, that can still effectively flush an intravenous catheter line, for example. In one particular embodiment, the cartridge is prefilled with 2.5 ml of saline. In some embodiments, the syringe may be offered in a complete line of syringes of different volumes. For example, the range of syringe sizes may include syringes that are capable of holding 1, 3, 6, 12, 15, 30, and/or 60 ml of an injectable liquid such as medicine in the proximal chamber. Each syringe size may have a common flush size in the distal chamber, for example 2.5 ml. Alternatively, each syringe size may include a distal chamber having a different volume.

As shown in FIGS. 20A and 20B, filling the distal chamber of the inner barrel 30 includes injecting a liquid, such as a flushing liquid, into the inner barrel 30 through the outlet of the outer barrel 10 and the conduit 44 of the inner barrel 30. The inner barrel is positioned at the proximal end of the outer barrel 10. As shown in FIG. 20A, the proximal end of the second piston 60 is positioned at the proximal end of the inner barrel 30. As the liquid 52 is injected into the inner barrel 30 of the cartridge, the volume of liquid 52 increases. The liquid pushes the proximal end 2003 of the piston 60 in the distal direction such that the distal chamber of the inner barrel 30 expands as it is filled with liquid 52, as shown in FIG. 20B. In some embodiments, the piston 60 may be pulled distally to assist in filling the cartridge chamber. In some embodiments, the inner barrel 30 will be filled until the locking mechanism is engaged and the proximal end of the piston 60 is locked with respect to the inner barrel 30.

As shown in FIGS. 20C and 20D, in some embodiments, the inner barrel 30 is filled independently from the outer barrel (not shown). The liquid 52 may be injected into the cartridge chamber via a needle or nozzle 2102 positioned within the conduit 44. Once filled, the inner barrel 30 may then be placed within the outer barrel, such that the inner barrel 30 is movable within the outer barrel. As shown in FIG. 20C, the proximal end 2103 of the piston 60 is positioned at the distal end of the inner barrel 30. As the liquid 52 is injected into the inner barrel 30 of the cartridge, the liquid 52 fills the volume of the chamber within the inner barrel, as shown in FIG. 20D. In this embodiment, the needle does not completely occlude the conduit 44 such that air is allowed to escape as the distal chamber of the inner barrel 30 is filled with fluid. Alternatively, the liquid 52 can move the proximal end 2103 of the cartridge plunger distally to expand the distal chamber as the inner barrel 30 is filled, as described above. These filling techniques could also be utilized when the cartridge is disposed (to start) within the outer barrel.

In an alternative embodiment, the distal chamber may be filled through the distal end of the inner barrel 30, which may be prior to inserting the second piston 60 into the inner barrel. In this embodiment, the conduit 44 may be temporarily occluded while the distal chamber is filled through the open distal end of the inner barrel 30. Once the distal chamber is filled, the piston 60 may be positioned within the inner barrel 30, and in some embodiments locked in place with respect to the inner barrel 30 by the locking mechanism. Once the distal chamber is closed off by the piston 60, the occlusion from the conduit 44 such that air trapped in the distal chamber during the filling and positioning of the piston 60 may escape.

Once a caregiver receives a syringe having a prefilled distal chamber, filling the proximal chamber (of the outer barrel 10) follows the standard operation for filling a syringe, which includes the steps of (1) fitting a syringe with a needle (metal or plastic) to penetrate the seal on a medicine bottle; (2) pulling the handle of the syringe back (distally) to draw air into the syringe of equal or greater volume than the medicine that is to be withdrawn; (3) inserting the air filled syringe with attached needle into the medicine bottle; (4) depressing (pushing proximally) the plunger to inject the air into the medicine bottle; (5) pulling the handle of the syringe back (distally) to draw medicine from the bottle into the syringe; and (6) withdrawing the needle/syringe from the medicine bottle and removing the needle from the syringe.

The syringe may then be connected to the patient or patient line at a luer port for injection of the medicine. The handle is depressed to inject the medicine, then the cartridge is unlocked and the handle depressed further to inject the saline. The syringe is removed and discarded.

In general, a method of using a syringe includes the steps of drawing a second liquid (such as medicine) into the outer barrel 10 through the proximal outlet by moving the inner barrel 30 distally within the outer barrel 10 and creating a second liquid-air interface within the conduit. As described above, the second liquid-air interface and the first liquid-air interface define a bubble which, cooperating with the conduit 44, prevent movement of the first liquid out of the conduit 44 and prevent movement of the second liquid into the conduit 44. In some embodiments, the method further includes the steps of (a) expelling the second liquid (such as medicine) from the outer barrel 10 through the proximal outlet by moving the inner barrel 30 proximally within the outer barrel 10, (b) releasing the locking mechanism from a locked configuration to an unlocked configuration to allow movement of the piston 60 within the inner barrel 30, and (c) expelling the first liquid (such as saline) from the inner barrel of the cartridge through the proximal outlet by moving the piston 60 proximally within the inner barrel 30. In some embodiments, the step (c) of expelling the first liquid includes expelling the gas bubble from the conduit, along with the first liquid, through the proximal outlet. As described above, the gas bubble within the conduit disposed between the first liquid-gas interface and the second liquid-gas interface is a small bubble, safe for injection into a patient.

The different stages of use described above will have varying effects on the bubble 1803 formed in the conduit 44. FIG. 21A represents the shape of the air pocket 2210 in the conduit 44 during shipping. As shown, only one liquid-air interface 1801 will be present, as no medicine or additional fluid will have been added to the proximal chamber.

FIG. 21B represents the shape of the air pocket 2210 in the conduit 44 when the proximal chamber is filled with air prior to ejecting the air into the bottle containing fluid (for later uptake of fluid into the proximal chamber). Although there is a slight negative pressure created in the proximal chamber as air is draw in from the open atmosphere, the effect is negligible, and the shape of the liquid-air interface 1801 remains substantially unchanged.

FIG. 21C represents the shape of the air pocket 2210 during injection of air into the bottle. The arrow 2112 shows the net direction of force on the air pocket 2210 resulting from pressure generated as the proximal chamber is depressed. There is a distal deformation of the liquid-air interface 1801 resulting from the force.

FIG. 21D represents the shape of the air pocket 2210 during withdrawal of medication into the proximal chamber from the bottle. The arrow 2114 shows the net direction of force on the air pocket 2210. The negative pressure in the proximal chamber causes a proximal deformation of the liquid-air interface 1801 without disrupting the liquid-air interface 1801.

FIG. 21E represents the air bubble 1803 formed after fluid has been filled into the proximal chamber. A net zero force is on the bubble 1803 such that the liquid-air interface 1802 formed near the proximal chamber has the same curvature in the opposite direction as the liquid-air interface 1801 formed near the distal chamber.

FIG. 21F represents the shape of the air bubble 1803 as the fluid from the proximal chamber is administered to the patient. As the fluid is released, the bubble 1803 experiences a net distal force as a result of the pressure in the proximal chamber, represented by the arrow 2116. As a result, both the liquid-air interface 1802 near the proximal chamber and the liquid-air interface 1801 near the distal chamber move distally.

FIG. 21G represents the shape of the air bubble 1803 after the contents have been dispensed from the proximal chamber. Due to the surface tension of the bubble, a small amount of liquid remains in conduit on the proximal side of the bubble 1803. As a result, the liquid-air interface 1802 on the proximal side remains intact.

FIG. 21H represents the shape of the bubble 1803 as the fluid 52 in the distal chamber is expelled. The net direction of force on the bubble 1803 is proximal, as shown by arrow 2118. As the solution 52 is discharged, there is initial proximal deformation of both the liquid-air interface 1802 on the proximal side as well as the liquid-air interface 1801 on the distal side. The force 2118 is great enough in the proximal direction that the bubble 1803 will eventually be displaced.

As discussed above, the syringe can include a locking mechanism to prevent movement of the piston 60 within the inner chamber 30. In some embodiments, as shown in FIGS. 22A and 22B, the syringe includes a locking mechanism 2200 having a flexible arm 2207 with a first end connected to the piston 60 and a free end having a tab 2210, and a groove 2211 configured to receive the tab 2210. In some embodiments, the flexible arm 2207 is coupled to the proximal end of the piston 60 while the groove is defined by the inner barrel 30. In some embodiments, the flexible arm 2207 of the locking mechanism may be coupled to the distal handle of the piston 60 as shown in FIG. 22A.

The geometry of the groove 2211 is such that it receives the tab 2210 and holds the tab 2210 in place, preventing movement of the piston 60 with respect to the distal chamber. The geometry of the groove 2211 is such that the tab 2210 can be moved in and out of the groove 2211 in the circumferential direction, i.e. by rotating the piston 60 with respect to the cartridge chamber. The tab 2210 cannot be moved in and out of the groove 2211 in the axial direction (i.e. distally or proximally). Once the tab 2210 is rotated out of the groove 2211, however, the locking mechanism is in the unlocked configuration, and the tab 2210 may be moved distally or proximally with respect to the groove, allowing the piston 60 to be moved distally or proximally with respect to the inner barrel 30.

The flexible arm 2207 is configured such that it has an equilibrium configuration and a bent configuration. In the equilibrium configuration, the tab 2210 extends beyond the outer circumferential surface of the piston 60. In the bent configuration, the free end of the flexible arm 2207 is bent inward such that the tab 2210 is within or flush with the outer surface of the piston 60. When the flexible arm 2207 is in the bent configuration, the piston 60 can move with respect to the inner barrel 30. When the flexible arm 2207 is in the equilibrium configuration, the tab is able to extend beyond the outer circumferential surface of the piston 60. It is in the equilibrium configuration that the tab 2210 will be received by the groove 2211 and that the locking mechanism 2200 is in the locked configuration. The flexible arm 2207 is biased toward the equilibrium configuration such that once the tab 2210 reaches a groove 2211, the tab 2210 will spring into the groove 2211, thereby locking the locking mechanism 2200. To release the tab 2210 from the groove 2211, the tab 2210 is rotated out of the groove 2211.

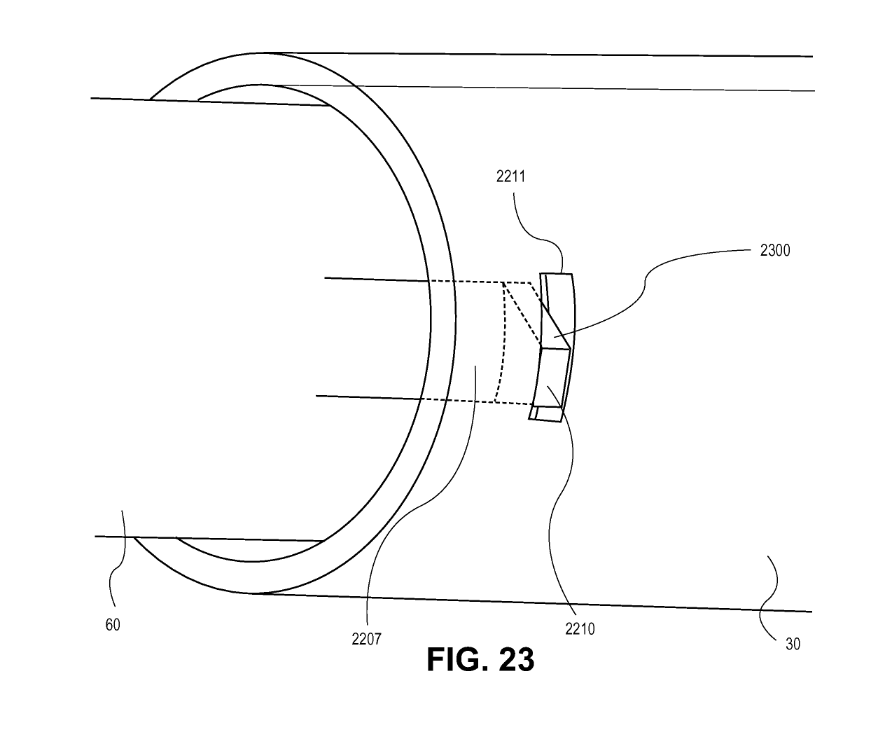

As shown in FIG. 23, the tab 2210 includes a single ramped surface 2300. As the piston 60 is rotated counter-clockwise (toward the top of the Figure), for example, the ramped surface 2300 interacts with the edge of the groove 2211 such that the groove pushes the tab down along the ramped surface 2300, allowing the flexible arm 2207 to transition from the equilibrium configuration to the bent configuration. Once the piston 60 is rotated sufficiently such that the tab 2210 is released from the groove 2211, the locking mechanism become unlocked, and the piston 60 may be moved proximally or proximally with respect to the inner chamber 30. In some embodiments, as shown in FIGS. 22A and 22B, the tab 2210 includes two ramped surfaces. Although shown as distinct ramped surfaces, the surfaces can be more continuous such that the tab is approximately semi-circular in shape. As a result of the ramped surfaces, the tab as shown in FIGS. 22A and 22B may be rotated in both the clockwise direction or in the counter-clockwise direction to release the locking mechanism. As shown in FIG. 23, the tab 2210 includes a single ramped surface 2300 and is therefore substantially trapezoidal shaped. The tab 2210 may include any suitable number of ramped surfaces and have any suitable geometry.

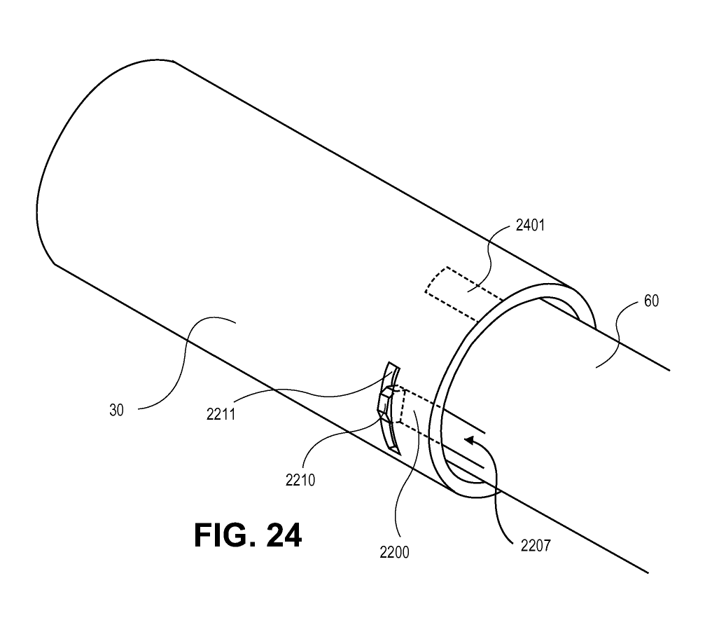

In some embodiments, as shown in FIG. 24, the syringe further includes an additional adjacent groove 2401 adjacent to the locking mechanism 2200. The adjacent groove 2401 is configured to receive the tab 2210 once it is rotated out of groove 2211 and when the locking mechanism 2200 is in the unlocked configuration. When the tab 2210 springs from the bent configuration back into a partial equilibrium configuration, it will be received by the adjacent groove 2401. This will signify that the locking mechanism 2210 is unlocked and that the piston 60 can be moved with respect to the inner barrel. Furthermore, when the tab 2210 is received by the adjacent groove 2401, the adjacent groove 2401 can give a tactile or audible signal to the user confirming that the release of the locking mechanism 2200 is complete. The adjacent grooves are located on the inner surface of the inner barrel 30. The adjacent grooves may include a tapered depth such that the adjacent groove permits movement (in the proximal direction) of the piston 60 with respect to the inner barrel 30. The tapered depth may function to slowly transition the flexible arm 2207 from the equilibrium configuration to the bent configuration as the piston 60 is moved proximally with respect to the inner barrel 30.

In some embodiments, the locking mechanism 2200 may include two grooves 2210 and two flexible arms 2207 on opposite sides of the inner barrel 30 from one another. In this embodiment, the syringe may include two adjacent grooves 2401 on opposite side from one another and about 90 degrees from the grooves 2211. The syringe may alternatively include any suitable number of locking mechanisms and adjacent grooves.

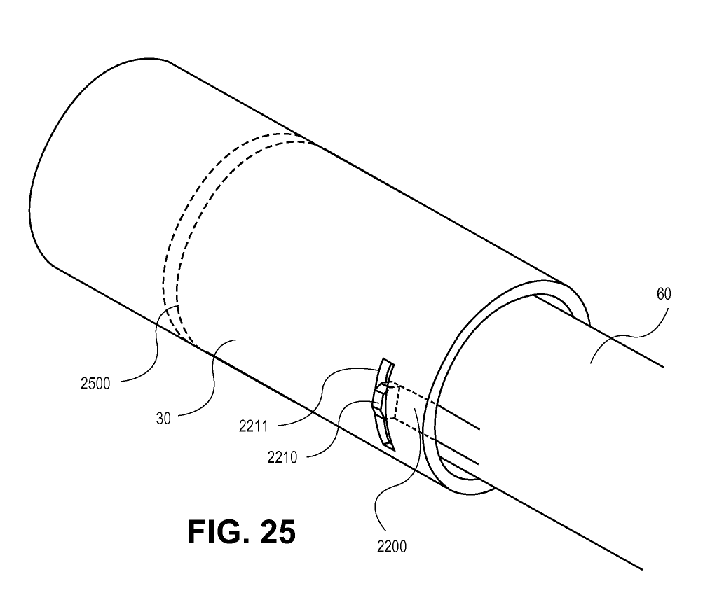

In some embodiments, as shown in FIG. 25, the syringe further includes an additional groove 2500 positioned on the inner surface of the inner barrel 30, proximal to the locking mechanism 2200. The groove 2500 is configured to receive but never release the tab 2210 as the proximal end of the piston 60 reaches the proximal end of the inner barrel 30. In some embodiments, the syringe may include a single additional groove 2500 or multiple additional grooves 2500 distributed around the circumference of the inner barrel 30. Alternatively, a single additional groove 2500 may extend all the way around the circumference of the inner barrel 30, as shown in FIG. 25. The geometry of the additional groove 2500 may be configured such that once the additional groove 2500 receives the tab 2210, the tab 2210 cannot be released from the additional groove 2500. This prevents the withdrawal of the piston 60 once the flush liquid has been expelled from the inner barrel 30 and prevents reuse of the syringe which could be harmful to patients.

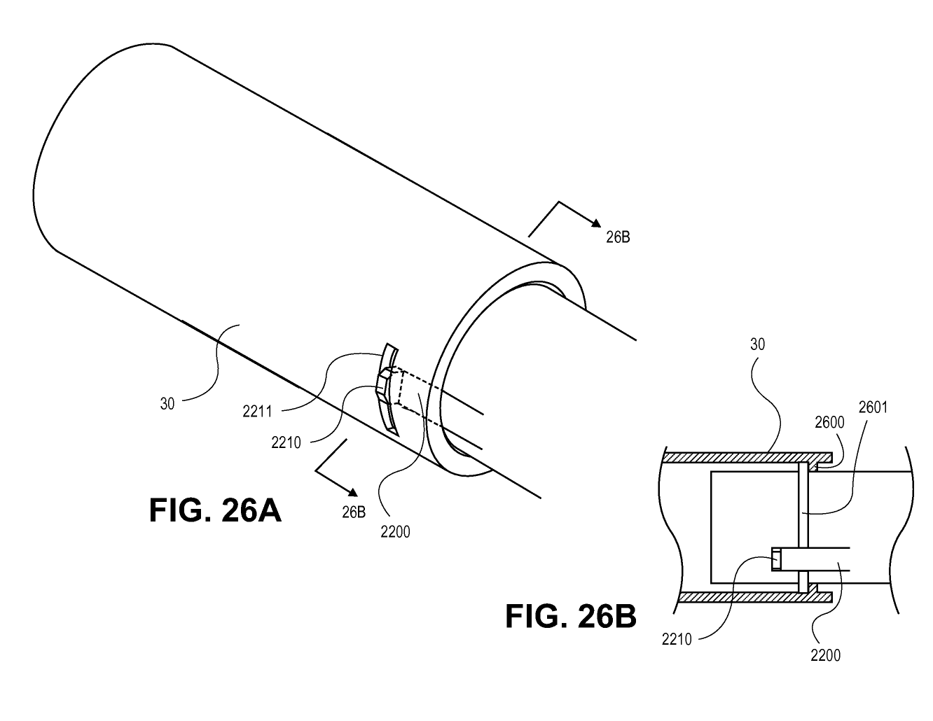

In some embodiments, as shown in FIGS. 26A and 26B, the syringe further includes a first ridge 2600 on the inner surface of the inner barrel 30. The ridge is configured to prevent the withdrawal of the piston 60 from the inner barrel 30 in the distal direction when the locking mechanism 2200 is in the unlocked configuration. In some embodiments, the syringe further includes a second ridge 2601 on the outer surface of the proximal end of the piston 60. The first and second ridges are configured such that the first ridge 2600 prevents movement of the second ridge 2601 in the distal direction, thereby preventing the withdrawal of the second end from the cartridge when in the unlocked position. The ridges may extend around the circumference of the syringe, as shown, or may only partially extend around the circumference of the syringe. Alternatively, the syringe may include a series of ridges distributed around the circumference of the syringe.

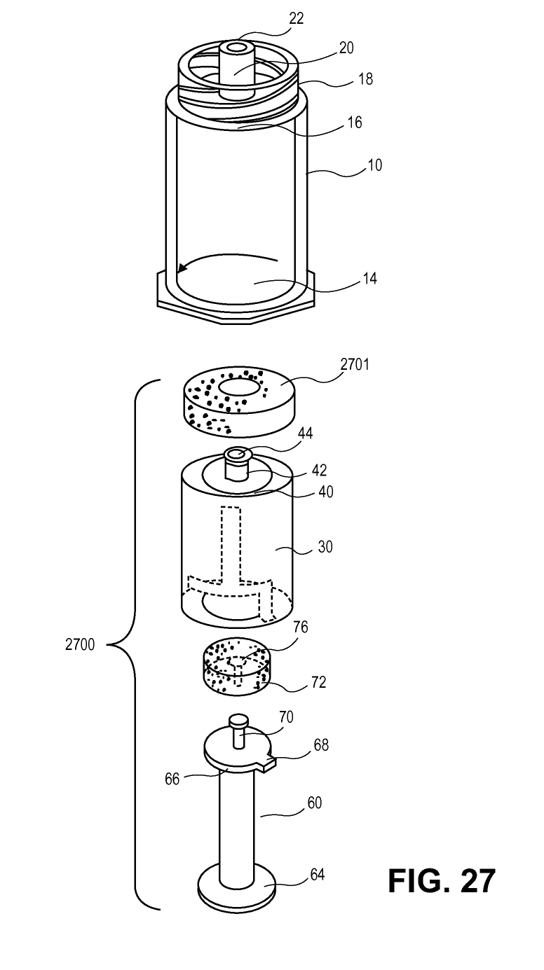

The syringe as described above may be configured such that the inner barrel 30 and the piston 60 are first moved together proximally within the outer barrel 10 to expel the liquid from the proximal chamber, such as medicine. Once the liquid is expelled from the proximal chamber, and the cartridge is positioned toward the proximal end of the outer barrel 10, the locking mechanism 2200 may be released by rotating the piston 60. While the piston 60 is rotated, the inner barrel 30 remains fixed with respect to the outer barrel 10. The inner barrel 30 may be fixed with respect to the outer barrel 10 in one of several variations. In a first variation, as shown in FIG. 27, inner barrel 30 is movable within outer barrel 10. As shown, outer barrel 10 has a non-circular cross section, such as an oval cross section. The inner barrel 30 of cartridge includes a proximal end 2701 having an outer diameter that is also non-circular (e.g. oval). As such, the inner barrel 30 is movable in the distal and proximal direction within the outer barrel 10. The inner barrel 30 cannot, however, rotate within the outer barrel 10 due to the non-circular cross sections. The inner diameter of the inner barrel 30 can have a circular cross section. The piston 60 and sealing ring 72 also have circular cross sections such that the piston 60 can be placed within the inner barrel 30 and is movable in the distal and proximal directions within the inner barrel 30. Further, the piston 60 has a circular cross-section and can therefore rotate within the inner barrel 30 due to the circular cross sections. Thus, the piston 60 can therefore be rotated within the inner barrel to unlock the locking mechanism 2200.

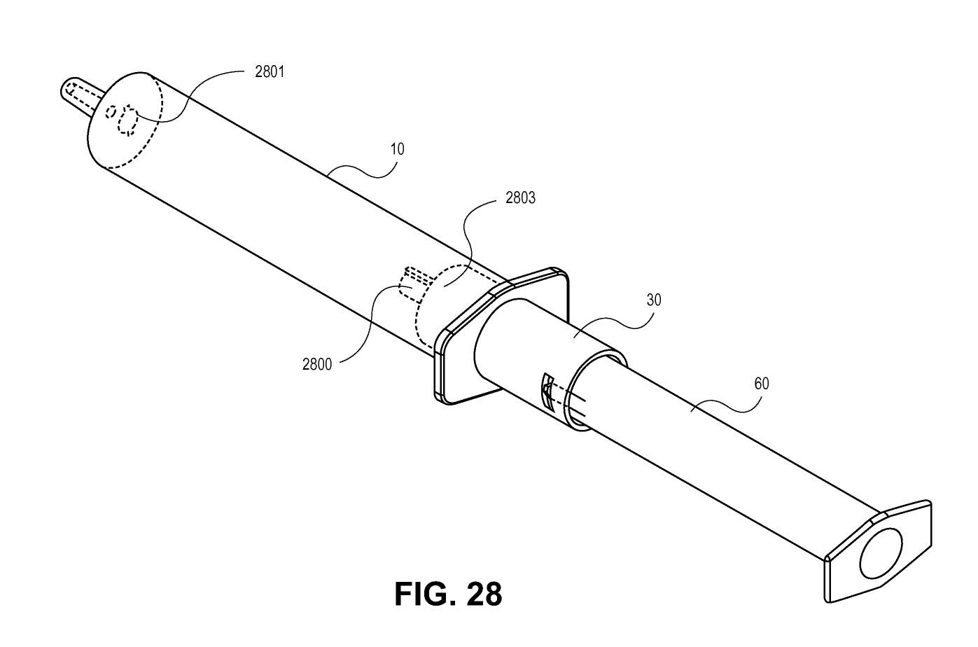

In a second variation, as shown in FIG. 28, the syringe includes a male/female locking mechanism comprising male portion 2800 and female portion 2801. Once the inner barrel 30 is moved proximally within the outer barrel 10 to expel the liquid from the proximal chamber and the inner barrel 30 is positioned toward the proximal end of the outer barrel 10, the male portion 2800 (coupled to the proximal 2803 of the cartridge) is moved into and received by female portion 2801 (coupled to the proximal end of the outer barrel 10). Once the male portion is fitted within the female portion, the male/female locking mechanism prevents rotation of the first end 2803 of the cartridge within the outer barrel 10. Therefore, the locking mechanism 2200 of the inner barrel 30 may be released by rotating the piston 60 with respect to the inner barrel 30 while the inner barrel 30 remains fixed with respect to the outer barrel 10.

In a third variation, the syringe may include a screw mechanism, such as a luer lock, such that the inner barrel 30 is screwed into the proximal end of the outer barrel 10 and locked into place to prevent further rotation.

In a fourth variation, there may be sufficient friction between outer surface of the inner barrel 30 and the inner surface of the outer barrel 10 such that as the piston 60 is rotated within the inner barrel 30, the inner barrel 30 remains fixed. This may be accomplished by having the inner barrel include a rubber stopper 72 (as shown in FIG. 1). The friction between the rubber stopper 72 and the inner surface of the outer barrel 10 can be sufficiently greater than the friction between the piston 72 and the inner barrel 30, such that as the piston 60 is rotated, the inner barrel 30 will not rotate with respect to the outer barrel 10. In such an embodiment the friction between the outer surface of the inner barrel 30 and the inner surface of the outer barrel 10 must remain appropriate for longitudinal movement of the cartridge.

In some embodiments, the syringe may further include lock state indicia that aid a user of the syringe by signifying when the locking mechanism 2200 is in the locked configuration and/or when the locking mechanism 2200 is in an unlocked configuration. The syringe may also bear warning not to prematurely rotate the piston 60 prior to the desired time of expelling the flush liquid, and/or any other suitable indication or warning. The lock state indicia may be printed onto a surface of the syringe or may be printed on a label coupled to the syringe. In the case of a label coupled to the syringe, the outer surface of the syringe may include a groove or recess sized to receive the label. As shown in FIG. 29, the syringe may include lock state indicia 2901 and 2902 that indicate when the locking mechanism is in the locked configuration and the unlocked configuration. As shown, the indicia 2901 (coupled to the inner barrel 30) and 2902 (coupled to the piston 60) do not line up when the locking mechanism 2200 is locked. As the piston 60 is rotated and the tab 2210 is released from the groove 2211, indicium 2902 will be moved to line up with indicium 2901 indicating that the locking mechanism is unlocked. As shown, the lock state indicia may be lines or other suitable symbols. The lock state indicia may alternatively include characters or words. For example, indicium 2902 may be the word “LOCKED” while indicium 2901 may be the prefix “UN” such that when the locking mechanism is unlocked and indicium 2901 is aligned with indicium 2902, together they spell the word “UNLOCKED”.

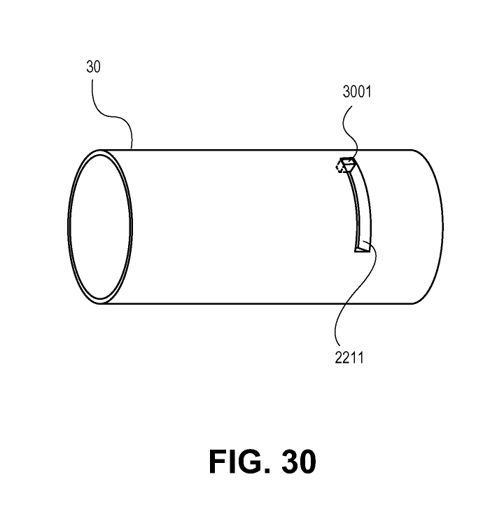

In some embodiments, as shown in FIG. 30, groove 2211 coupled to the inner surface of the inner barrel 30 of the cartridge may include a ridge 3001. The ridge 3001 is sized and configured to prevent the tab (not shown) of the locking mechanism from reentering the groove. For example, as shown, tab 2210 would be rotated out of groove 3000 by rotating the tab 2210 in the counter-clockwise direction (toward the bottom of the Figure). If one were to continue to rotate the tab 2210 in the counter-clockwise direction, the tab 2210 could reenter the groove 2211 from the opposite side, thereby returning the locking mechanism 2200 to the locked configuration. In some instances, this may not be desirable and therefore ridge 3001 may function to stop the tab 2210 from reentering the groove 2211 from that side, thereby maintaining the locking mechanism 2200 in the unlocked configuration.

One skilled in the art will appreciate that the present invention can be practiced by other than the preferred embodiments, which are presented for purposes of illustration and not of limitation.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A syringe comprising:
a cartridge comprising:
a first chamber,
a second end, the second end movable within the first chamber between a first position and a second position, and
a locking mechanism having a locked configuration and an unlocked configuration, the locking mechanism preventing movement of the second end within the first chamber while in the locked configuration, wherein the locking mechanism comprises:
a flexible arm having a first end coupled to the second end of the cartridge and a second free end,
a tab coupled to the second free end, and
a groove in the inner surface of the first chamber configured to receive the tab; and
a second chamber, wherein the cartridge is movable within the second chamber.

2. The syringe of claim 1, wherein the tab includes a ramped surface such that when the second end is rotated within the inner surface of the cartridge, the ramped surface allows the tab to slide more easily out of the groove.

3. The syringe of claim 2, wherein the tab includes two ramped surfaces such that the second end may be rotated in two directions within the first chamber.

4. The syringe of claim 1, further comprising an adjacent groove adapted to receive the tab when the locking mechanism is in the unlocked configuration.

5. The syringe of claim 4, wherein the adjacent groove includes a tapered depth such that the adjacent groove permits movement of the second end within the first chamber.

6. The syringe of claim 1, wherein the tab is substantially trapezoidal shaped.

7. The syringe of claim 1, wherein the tab is substantially semi-circular shaped.

8. The syringe of claim 1, further comprising a second groove configured to receive the tab when the second end is in the second position.

9. The syringe of claim 8, wherein the second groove extends around the circumference of the cartridge.

10. The syringe of claim 1, further comprising a first ridge on the inner surface of the cartridge, wherein the ridge is configured to prevent the withdrawal of the second end from the first chamber.

11. The syringe of claim 10, further comprising a second ridge on the outer surface of the second end of the cartridge and a second ridge, wherein the first ridge is configured to engage with the second ridge such that withdrawal of the second end from the first chamber is prevented.

12. The syringe of claim 1, wherein the cartridge further comprises a first end that defines a conduit in liquid communication with the first chamber.

13. The syringe of claim 12, wherein the first end of the cartridge is coupled to the first chamber such that when the second end of the cartridge is rotated within the first chamber, the first end is not rotated.

14. The syringe of claim 12, wherein the first chamber has a noncircular cross section.

15. The syringe of claim 1, further comprising indicia that signify when the locking mechanism is in the locked configuration and when the locking mechanism is in an unlocked configuration.

16. The syringe of claim 1, further comprising a ridge coupled to an end of the groove, wherein the ridge is configured to prevent the tab from reentering the groove after it has been released.

17. The syringe of claim 1, wherein the second end of the cartridge further comprises a handle sized and configured to move the second end within the first chamber.

18. The syringe of claim 1, further comprising an additional groove on an inner surface of the first chamber, the additional groove configured to receive and not release the tab. 19. A method of using a syringe having a cartridge having a first chamber, a second end movable within the first chamber, and a locking mechanism, and wherein the syringe further includes a second chamber having an outlet, wherein the method comprises:
expelling a liquid from the second chamber through the outlet by moving the cartridge within the second chamber toward the outlet;
rotating the second end of the cartridge with respect to the first chamber to release the locking mechanism; and
expelling a second liquid from the first chamber through the outlet by moving the second end of the cartridge within the first chamber toward the outlet.

19. The method of claim 18, wherein the second end includes a tab having a ramp, and wherein rotating comprises rotating the tab against a groove defined by the inner wall of the first chamber such that the tab is released from the groove.

20. The method of claim 19, wherein the rotating step further comprises further rotating the tab after the tab is released by the groove, such that the tab is received by a second adjacent groove.

21. A syringe comprising:
a cartridge comprising:
a first chamber,
a first end having a conduit in liquid communication with the first chamber,
a liquid disposed within the first chamber, wherein the conduit is adapted and configured to rely on a property of the liquid to prevent movement of the liquid out of the first chamber, and
a second end, movable within the first chamber;
a second chamber, wherein the cartridge is movable within the second chamber; and
a removable end cap coupled to both an outlet of the second chamber and the conduit.

22. The syringe of claim 21, wherein the end cap comprises an end surface at or within the conduit, and wherein the syringe is further adapted and configured such that the end surface, cooperating with the conduit, prevents movement of the liquid out of the first chamber.
